(19) United States
(12) Patent Application Publication
(10) Pub. No.: US 2016/0297828 A1
(43) Pub. Date: Oct. 13, 2016

(54) SPIROCYCLIC COMPOUNDS CONTAINING SPIRO[INDOLYL-3,1'-PYRROLO[3,4-C]PYRROLE] CORE AND SULPHUR-CONTAINING AMINO ACID RESIDUES

(71) Applicant: Gleb Vladimirovich ZAGORII, Kiev (UA)

(72) Inventor: Gleb Vladimirovich Zagorii, Kiev (UA)

(21) Appl. No.: 15/100,543

(22) PCT Filed: Dec. 3, 2014
(86) PCT No.: PCT/UA2014/000127

§ 371 Date: May 31, 2016

(30) Foreign Application Priority Data
Dec. 3, 2013 (UA) A 2013 14072

(51) Int. Cl. C07D 487/20; C07C 55/10; C07D 307/62

(52) U.S. Cl. CPC C07D 487/20; C07D 307/62; C07C 55/10

(57) ABSTRACT

The present invention relates to spirocyclic compounds on the basis of 2-oxindole derivatives containing a spiro[indolyl-3,1′-pyrrolo[3,4-c]-pyrrole] core and biogenic sulphur-containing amino acid residues, which display a glucocorticoid-mimicking action by influencing 11β-HSD1 enzyme cortisone->cortisol conversion, or by inhibiting GRs- or GITR- or mineralocorticoid receptors, or other targets, but do not interfere with steroidal haemostasis in HPA; and compositions containing same and their use for therapy as part of undifferentiated stroke therapy (in the absence of final verification of the stroke subtype) at various stages of acute ischemic stroke (AIS), during the period of recovery from stroke and craniocerebral trauma, in patients with chronic cerebrovascular pathology (against a background of diabetes), in combinational therapy for Alzheimer's disease and encephalopathy of various origin (discirculatory, alcoholic, infectious-toxic), and diabetes, combinational therapy for retinal degenerative eye diseases, as part of combinational therapy for metabolic syndrome (obesity, in patients suffering from Cushing's syndrome, Reaven metabolic syndrome (also known as syndrome X or insulin resistance syndrome) and other diseases where GCs hormones play a key role.

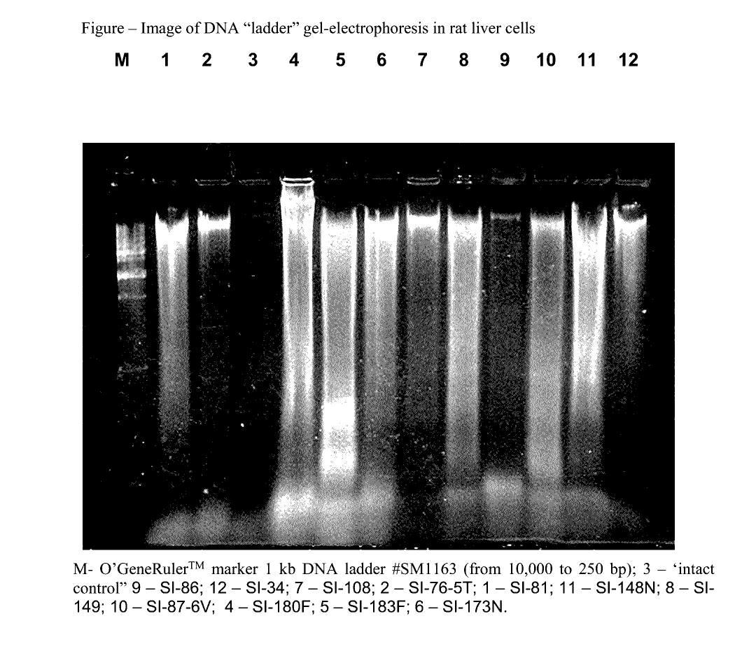

### CROSS REFERENCE TO RELATED APPLICATIONS

This application is a national stage application, filed under 35 U.S.C. §371, of International Application No. PCT/UA2014/000127, filed Dec. 3, 2014, which claims priority to Ukrainian Application No. a 2013 14072, filed Dec. 3, 2013, the contents of both of which as are hereby incorporated by reference in their entirety.

### BACKGROUND

This invention relates to pharmacy, namely to the design of novel pharmaceutical agents—compounds based on 2-oxindole derivatives, comprising spiro[indolo-3,1′-pyrrolo[3,4-c]pyrrol] core and remainders of biogenic sulphur-containing aminoacids, exhibiting glucocorticoid modulating activity by action on 11β-hydroxysteroiddehydrogenase (11β-HSD1) enzyme, responsible for cortisone→cortisol conversion, or by suppressing of glucocorticoid-(GRs), or glucocorticosteroid-TNF-induced-(GITR), or mineralocorticoid receptors or other targets, but without effect on steroid gaemostasis in hypothalamus-pituitary-adrenal system (HPA) and compositions containing thereof, and use thereof for treatment of diseases, pathogenesis of which is dramatically affected by glucocorticoid hormones.

Recently the attention of scientists was drawn to the search of selective inhibitors of 11β-HSD1 enzyme, which is a key factor in peripherical conversion of non-active cortisone into cortisol in humans (or 11-dehydro-corticosterone into 11β-corticosterone in rodents and some other higher mammals) in cells. As increased expression of this enzyme is important part of pathogenesis in series of diseases (diabetes mellitus, metabolic syndrome (adiposity, patients with Cushing syndrome, metabolic Riven syndrome (also known as X syndrome or syndrome of insulin resistance), impaired glucose tolerance, increased level of plasma triglycerides, insulin resistance, arterial hypertension, chronic subclinical inflammation, thromboses, stroke and some other cardiovascular diseases), then reduced activity of said enzyme can be used for therapy of these diseases [C. Fotsch and M. Wang, J. Med. Chem., 51, 4851-4857 (2008)].

It is well established that pathogenetically 11β-HSD1 is activated in adipose tissues in humans and rodents having adiposis (Livingstone et al. (2000) Endocrinology 131: 560-563; Rask et al. (2001) J. Clin. Endocrinol. Metab. 86: 1418-1421; Lindsay et al. (2003) J. Clin. Endocrinol. Metab. 88: 2738-2744; Wake et al. (2003) J. Clin. Endocrinol. Metab. 88: 3983-3988). Increased activity of 11β-HSD1 in these mice (2-3-fold) is very similar to that observed in humans having obesity (Rask et al. (2001) J. Clin. Endocrinol. Metab. 86: 1418-1421). This suggests that local transformation, mediated by 11β-HSD1, of non-active glucocorticoid into active one, would effect in a complicated way, on sensitivity to insulin of the whole body. Thus the blockage of 11β-HSD1 could result in increasing of insulin sensitivity and tolerance and reducing of glucocorticoid excitotoxicity in brain [Kato et al (2013) Front Integr Neurosci. 7:53].

It is also known that glucocorticoids inhibit insulin secretion (contrinsulinic action), stimulated by glucose in pancreatic beta-cells [Billaudel and Sutter (1979) Horm. Metab. Res. 11: 555-560]. Also, both in rodents with Cushing syndrome and in fa/fa Zucker rats having diabetes, glucose-stimulated insulin secretion is notably reduced [Ogawa et al. (1992) J. Clin. Invest. 90: 497-504]. Therefore we suggest that suppression of 11β-HSD1 enzyme would be useful for pancreatic gland, including enhancing of glucose-stimulated insulin release.

In the light of experimental data indicating to relationship between diabetes mellitus of the 2-nd type and metabolic syndrome (mC) pathogenesis, and also 11β-HSD1 function in these pathological states [C. Day, Diabetes Vase. Dis. Res., 4, 32-38 (2007); R. H. Eckel, S. M. Grundy and P. Z. Zimmet, Lancet, 365, 1415-1428 (2005)], also associated with glucocorticoids, in particular, hypertension, obesity, insulin resistance, hyperglycemia, hyperlipidemia, excess of androgenic hormones (hirsutism, menstrual disorder, hyperandrogenism) and polycystic ovarian disease (PCOS), therapeutic agents, directed to intensification or suppression of these metabolic pathways by modeling of signal glucocorticoid transduction at the 11β-HSD1 level, are desirable.

11β-HSD1 enzyme is expressed predominantly in organs and tissues having high sensitivity to glucocorticoids, in particular, in liver, adipose tissues, lungs, CNS, aortal endothelium [J. R. Seckl and B. R. Walker, Endocrinology, 142 (4), 1371-1376 (2001); M. Wamil and J. R. Seckl, Drug Discovery Today, 12 (13/14), 504-520 (2007)], while 11β-HSD2 is activated in organs-targets of mineralocorticoids—in kidneys, intestine, salivary glands, placenta, vascular endothelium [P. M. Stewart and Z. S. Krozowski, Vitam. Horm., 57, 249-324 (1999)]. Additionally, in humans the expression of 11β-HSD1 in adipocytes correlates with adiposity degree and is independent on genetic factors. It was supported by studies of activity of said enzyme in monozygotic twins, one of which has suffered from adiposis, and another one had normal stature [K. Kannisto, K. H. Pietilainen, and E. Ehrenborg et al., J. Clin. Endocrinol. Metab., 89, 4414-4421 (2004)]. At obesity in liver the function of 11β-HSD1 is suppressed resulting in reducing of glucocorticoid concentration, decreasing of gluconeogenesis and adipogenesis. Probably this is protective factor preventing the accumulation of body weight and development of glucose intolerance [W. Artl and P. M. Stewart, Endocrinol. Metab. Clin. North. Am., 34, 293-313 (2005)]. But this adaptation mechanism of 11β-HSD1 activity attenuating in liver and therefore decreasing of cortisol production, absent at DM of the 2-nd type accompanied by adiposis, and increasing of glucocorticoid level can contribute to disease pathogenesis. In such a case adipocytes can be considered as primary and hepatocytes as secondary cell target for potential agents effecting on insulin resistance [P. M. Stewart, A. Boulton, and S. Kumar et al., J. Clin. Endocrinol Metab., 84, 1022-1027 (1999)]. In MC, increased activity of 11β-HSD1 in adipose tissues causes the local excess of cortisol and insulin resistance.

Also it was reported about the existence of a number of corticosteroid receptors located in neurons of hippocampus, hypothalamus and cerebral cortex (De Kloet et (1998) Endocr Rev. 19(3): 269-301). Corticosteroids are able to penetrate through hematoencephalic barrier and associate in brains with two receptor types—to gluco—and mineralocorticoids, respectively. The receptors to mineralocorticoids tend to affect via cell excitability increasing, while glucocorticoid receptors (GRs) have an inhibitory effect on neuronal activity and steroid-mediated control of neuron excitability is essential for information processing in brains. Corticosteroid receptors significantly effect on function of hippocampus and structures directly involved in formation of mood, memory and control on function of hypothalamus-pituitary-adrenal system (HPA). The importance of HPA axis in control of glucocorticoid concentration is obvious based on the fact that homeostasis disturbance in HPA loop at either excessive or insufficient secretion or action results in Cushing syndrome or Addison disease, respectively (Miller and Chrousos (2001) Endocrinology and Metabolism, eds. Felig and Frohman (McGraw-Hill, New York), 4th Ed. 387-524).

Additionally, chronical effect of high levels of glucocorticoids results in cognitive disturbances and is the manifestation of aging associated with progression of dementia (Wyrwoll et al (2011) Front Neuroendocrinol. 32(3): 265-286.). Both in old animals and in elderly humans the reduction of general cognitive functions is associated with an effect of glucocorticoids and 11β-HSD1 expression level (Alasdair M. J. MacLullich (2012), Neurobiology of Aging 33(1): 207-207). In elderly humans with chronically high cortisol level the decreasing of hippocampus neurons density and development of hippocampus athrophia are observed (Bauer (2005) Stress. 8(1): 69-83). The age-associated increasing of glucocorticoids accompanied by decrease in decreasing of threshold hippocampus neurons exciting, causing disruption of the consolidation of memory in aged rats. The similar situation occurs in neurodegenerative diseases (e.g. Alzheimer's disease) and is accompanied by a decrease in cognitive and memory functions (McCormick and Mathews (2010) Prog Neuropsychopharmacol Biol Psychiatry. 34 (5): 756-65). The treatment of primary hippocampal cells with carbenooxolone that is 11β-HSD1 inhibitor, protects cells from glutamate neurotoxicity exacerbation mediated by glucocorticoids (Rajan et al. (1996) J. Neurosci. 16: 65-70). Additionally, it was found that genetic deficiency of 11β-HSD1 in mice protects from associated with glucocorticoids hippocampal age-associated dysfunction (Yau et al. (2001) Proc. Natl. Acad. Sci. 98: 4716-4721). So it is considered that the inhibition of 11β-HSD1 will weaken the effect of glucocorticoids in the brain and protect its tissue from the harmful effects of glucocorticoids on neuronal function, including cognitive impairment, dementia and/or depression.

Thus, in experimental diabetes mellitus and acute cerebrovascular accident, prescription of metyrapon (nonsteroid blocker of steroid 11β-hydroxylase) in CA1 hippocampus area and somato-sensory cortex of rats, the decrease is observed in density of destructive-modified neurons along with area retentions well as density of morphologically intact neurocytes, and protects against ischemia and excitotoxically-induced brain damage in rodents [Drouet (2012) Eur J Pharmacol. 5, 682 (1-3): 92-8]. Elevated levels of corticosterone in rat brains during hypobaric hypoxia causes neurodegenerative changes associated with effect on central GRs, whereas GRs inhibition may provide therapeutic effect in improving of induced memory impairment on the background of hypobaric hypoxia [Baitharu et al (2013) Behav Brain Res. 240: 76-86]. The administration of metyparon from the 3rd to the 7th day on the background of hypobaric hypoxia in rats allows to eliminate increased corticosterone level induced by hypoxia, and leads to reduced lipid oxidation, neurodegeneration and improved intracellular energy metabolism. Additionally, the administration of exogenous corticosterone along with metyrapon in hypoxia reduces the neuroprotective metyrapon effect, indicating to corticosterone role in mediating neurodegeneration and memory impairment induced by hypobaric hypoxia [Schaaf et al (2000) Stress. 3 (3): 201-208. Review; Baitharu et al (2012) Behav Brain Res. 228 (1): 53-65]. The use of metyrapon or glucocorticoid receptor antagonists (GRA) and progesterone receptor antagonists (PRA)-RU38486 (mifepristone) or non-peptide glucocorticoid receptor antagonist of type 1 (CRH-R1) R121919 though confirmed the prospectively of glucocorticosteroid level correcting for neuroprotection, but their clinic use is not appropriate because they impair homeostasis in HPA [Belda et al (2012) Horm Behav. 62(4): 515-524; Bluthgen et (2013) Aquat Toxicol. 144-145C: 96-104].

The first and the most well-studied exogenous non-selective 11β-HSD inhibitor of plant origin are triterpenoids (sapogenin)—glycyrrhetic acid and diglucoronide thereof—glycyrrhizic acid contained in the roots rhizomes of Glycyrrhiza glabra L. and G. uralensis F [G. A. Tolstikov, L. A. Baltina, N. G. Serdiuk, Chemical. Pharm. Zh., 8, 5-14 (1998)]. Glycyrrhetic acid hemisuccinate (carbenoxolone), known since the mid 50's of the last century as antiulcer agent, has also shown in experiments on mast mice for its effective reduction of insulin and lipids in plasma [A. M. Nuothio-Antar, D. L. Hachey, and A. H. Hasty, Am. J. Physiol.: Endocrinol. Metab., 293, E1517-E1528 (2007).]. In healthy volunteers and patients with type 2 DM the use of carbenoxolone improves liver insulin sensitivity and causes neuroprotective effect on ischemic stroke models due to low glucocorticoids production in the brain [Beraki et al (2013) PLoS ONE 8 (7): e69233]. In two placebo-controlled crossover studies the carbenoxolone administration increased the speech rate and verbal memory (Sandep et al. (2004) Proc. Natl. Acad. Sci. Early Edition: 1-6). However, the drug was not used as a clinical antidiabetic agent due to its ability not only to inhibit 11β-HSD1 but to lower 11β-HSD activity, resulting in excess of renal mineralocorticoids and, consequently, to reabsorpthion of sodium ions, to hypokalemia and hypertension. Additionally, carbenoxolone is characterized by low lipophilicity as it poorly penetrates to adipose tissue—site of main 11β-HSD1 expression [K. A. Hughes, S. P. Webster and V. R. Walker, Expert Opin. Investig. Drugs, 17 (4), 481-496 (2008).].

Recently the pathogenetic feasibility of blocking tissue glucocorticosteroid activity was shown by administration of experimental compounds GRA-CORT 108 297 or LLY-2707 [Belanoff et al (2011) Eur J Pharmacol. 655 (1-3): 117-120; Belanoff et al (2012) Diabetes Obes Metab. 12 (6): 545-7; Sindelar et al (2013) J Pharmacol Exp Ther. 25: 1-25] for treatment of metabolic syndrome similar to diabetes mellitus and weight gain induced by the use of atypical antipsychotic agents (AAPDs), for example, olanzapine. The similar positive effect on reducing rat weight was observed when using RU38486 (mifepristone) on the background of the HPA homeostasis glucocorticosteroids disorders caused by olanzapine (Bebe et al (2006) Behav Brain Res. 171(2): 225-229).

EP2540723A1, WO 2004/089470, WO 2004/089896, WO 2004/056745 and WO 2004/065351 disclose the 11β-HSD1 inhibitors of non-steroid structure based on amides of different structure. Additionally 11β-HSD1 inhibitors which are non-steroid structures are reported in US 2005/0282858, US 2006/0009471, US 2005/0288338, US 2006/0009491, US 2006/0004049, US 2005/0288317, US 2005/0288329, US 10 2006/0122197, US 2006/0116382 and US 2006/0122210), INCY0035 (US 2007/0066584). The closest in structure to compounds presented herein are analogs thereof based on 2-oxindoleo-spiropiperidine amides (US 20080306102 A1), however, the authors did not indicate their cerebroprotective, cytoprotective, antioxidant, antyhypoxia, antidiabetic properties and toxicity level, in addition compounds comprising spiro [indolinon-3,4′-piperidine]moiety have the potential to cause adverse effects on the nervous system, particularly intrinsic for substances with related thereto structure of natural alkaloids that exhibit toxic properties in relation to nervous conduction, such as surogatoxin, prosurogatoxin and neosurogatoxin from clam (Babylonia japonica) and have holino- and adrenoblocking action [Ayajiki et al (1998) Jpn J Pharmacol. 78 (2): 217-23], and also are very similar in chemical structure to the substance described previously as a local anesthetic agent [Kornet M J, Thio A R (1976) J. Med. Chem 19 (7): 892-898].

The first phase of clinical trials of another non-steroid 11β-HSD1 inhibitor-fluorinated tiazolon (AMG-221) confirmed its good tolerability and suppressing activity on 11β-HSD1 in patients with adiposis. The second stage of AMG-221 studies was launched in 2007, but two years later, developers still have positioned it as a substance that is on the first phase of clinical trials [S. P. Webster and T. D. Pallin, Expert Opin. Ther. Patents, 17 (12), 1407-1422 (2007)]. In addition, some of the proposed 11β-HSD1 inhibitors are not active enough compared with proposed in this patent compounds. Thus, compounds of non-steroid nature BVT-2733 (3-chloro-2-methyl-N-(4-(2-(4-methylpiperazin-1-yl)-2-oxoethyl)thiazol-2-yl)benzenesulfonamide hydrochloride-specific inhibitor 11β-HSD1) when administered 3 and 7 hours after reperfusion at doses of 60 mg/kg and 30 mg/kg reduced the amount of ischemic brain damage in rats (Beraki et al (2013) PLoS ONE 8 (7): e69233). However, the compound requires administration in 3-6 times higher doses than compounds presented herein. Moreover, its properties to apoptosis are not known.

Another promising direction for use of 11β-HSD1 inhibitors is the treatment of glaucoma, as aqueous ocular humor is produced in uncoated epithelial cells (UEC), and flows through the trabecular network cells. 11β-HSD1 is localized in the UEC cells (Stokes et al. (2000) Invest. Ophthalmol. Vis. Sci. 41: 1629-1683; Rauz et al. (2001) Invest. Ophthalmol. Vis. Sci. 42: 2037-2042) and its function is probably to increase glucocorticoid activity in these cells. This is confirmed by the fact that free cortisol concentration is significantly higher than cortisone concentration in ocular watery moisture (14:1 ratio). The functional importance of 11β-HSD1 in the eye is evaluated using the inhibitor-carbenooxolon in healthy volunteers (Rauz et al. (2001) Invest. Ophthalmol. Vis. Sci. 42: 2037-2042). Seven days after treatment with carbenooxolon intraocular pressure (IOP) was decreased by 18%. Thus, one can assume that inhibition of 11β-HSD1 in the eye would reduce local concentration of glucocorticoids and IOP, providing beneficial effect in the treatment of glaucoma.

### DETAILED DESCRIPTION

Thus, there is continuing need for new and improved medicines that have glucocorticostimulating activity, including antidiabetic, neuroprotective effect in non-differentiated treatment of stroke (without final verification of its subtypes) in different periods of cerebrovascular accident (CVA), in recovery period after stroke and traumatic brain injury in patients with chronic cerebrovascular pathology (on the background of diabetes mellitus), adjuvant therapy for Alzheimer's disease and encephalopathies of various origins (circulatory, alcohol, infectious-toxic), adjuvant therapy for diabetes mellitus, retinodegenerative eye diseases, in part of complex treatment of metabolic syndrome, adiposity, patients with Cushing syndrome, metabolic Riven syndrome (also known as X syndrome or syndrome of insulin resistance) and other diseases. As expected, these therapeutic agents will decrease hydrocortisol concentration acting on enzyme conversion of cortisone→cortilol-11β-HSD1, or GRs-suppression, or GITR, or other targets, but without interfering with steroid hemostasis.

The inventors have set themselves the task to develop compounds and pharmaceutical compositions, which would be benefit to meet these needs.

The set task is solved by development of spirocyclic compounds based on derivatives of 2-oxindole, comprising spiro[indolo-3,1′-pyrrolo[3,4-c]-pyrrol] core and remainders of biogenic sulphur-containing aminoacids of general Formula I.

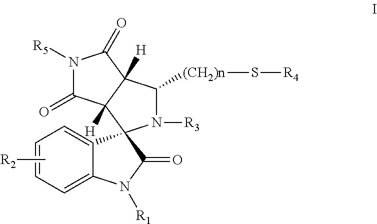

wherein:

### R₁ is H, Me-, Et-, Allyl-, -Bn;

R₂ is H, 5-Me, 5-F, 5-Br, -5-OCF₃, 5-NO₂;

### R₃ is H or —N═O;

R₄ is residuals of biogenic sulphur-containing aminoacids, selected from methionine (n=2, R₄=Me), ethionine (n=2, R₄=Et), cysteine (n=1, R₄=H) or cysteine alkyl-derivatives, wherein R₄=Bn or —CH₂CO₂Et, or Alyl-;
R₅ is H, or remainders of Ar, wherein Ar is p-Tolyl, m-Tolyl, 2-(HO)Ph-, 3-(HO)Ph-, 4-Br-Ph-;
4-NO₂-Ph-; 2-NO₂-Ph-; 2-Br-Ph- or 4-(HOOC)Ph-,
and pharmaceutically acceptable salts thereof of the Formula II

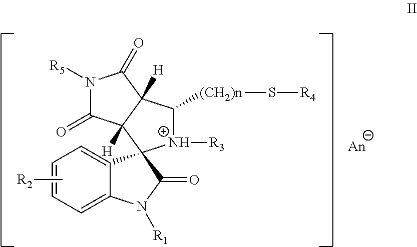

wherein An⁻ is selected from the group consisting of chloride, bromide, iodide, succinate, hemisuccinate, L-aspartate, tartrate or hydrotartrate, nicotinate, L-ascorbate, maleate or hydromaleate, fumarate, hydrofumarate, citrates, L-lactate, L-malate, phosphate, sulphate, benzoate, acetate, pivolate, glutarate, glutamate, asparaginate. And also corresponding solvates, hydrates, enantiomers etc. thereof.

Said compounds show glucocorticoid-modeling activity by action on 11β-HSD1 enzyme of cortisone→cortisol conversion or GRs-suppression or GITR-receptors or other targets, but without interfering with steroid hemostasis in HPA and also exhibit antioxidant, antihypoxic, cerebroprotective and cytoprotective effect.

According to one of embodiments this invention provides the use of above compounds for treatment of diseases pathogenesis of which plays a key role in increased cortisol production. In particular, for treatment of any of the following diseases or any combination of two or more of the following diseases: for adjuvant therapy for diabetes mellitus, as a part of non-differentiated stroke therapy (without final verification of its subtypes) in different CVA periods, in recovery period after stroke and traumatic brain injury, in patients with chronic cerebrovascular pathology (including on the background of diabetes mellitus), for adjuvant therapy of Alzheimer's disease and encephalopathies of various origins (circulatory, alcohol, infectious-toxic), retinodegenerative eye diseases, as part of complex treatment of metabolic syndrome (adiposity, patients with Cushing syndrome, metabolic Riven syndrome (also is known as X syndrome or syndrome of insulin resistance) and other diseases, insulin resistance, hyperglycemia, hypertension, hyperlipidemia, cognitive impairment, depression, dementia, glaucoma, cardiovascular disease; osteoporosis; inflammation, metabolic syndrome; excess of androgenic hormones or polycystic ovary syndrome (PCOS).

In accordance with another embodiment this invention provides the use of compounds of general Formula I or II for manufacture of pharmaceutical compositions in combination with pharmaceutically acceptable excipients that ensure their use as finished drugs as appropriate dosage forms such as tablets, pills, powders, lozenges, sachets, suspensions, emulsions, solutions for oral administration, syrups, aerosols (as a solid or in liquid medium), ointments, drops, soft and hard gelatin capsules, suppositories, injection solution s, and infusions.

Additionally, these compounds are provided to develop drugs effective in a single dose of 0.25 to 50 mg/kg (dosage frequency depending on the disease, but not more than 200 mg/kg).

The invention also provides a process for preparation compounds of general Formula I and by two-stage synthesis based three-component enantioselective condensation reaction, which is one-stage condensation of the corresponding pyrrol-2,5-dione with 1H-indole-2,3-dione and biogenic sulfur-containing amino acids in polar solvent media mixed with water. Suitable solvents are in particular methyl or isopropyl or ethyl alcohol or acetonitrile, used in a mixture with water at the ratio of 2:1 to 10:1. In particular, the most appropriate is the process for preparation these compounds, wherein the most preferred ratio of organic solvent and water is the ratio of 3:1.

According to another embodiment this invention provides a process for preparation of salts of general Formula II, which consists in dissolution of corresponding base of compounds of the Formula I in ethanol or a mixture of ethanol and water, or butanol, and adding aqueous or alcoholic solution of corresponding organic or inorganic acid of the Formula II, followed by evaporation in vacuum.

Additionally, this invention relates to compositions comprising compounds of the Formula (I) or pharmaceutically acceptable salt of the Formula II and at least one pharmaceutically acceptable carrier.

Additionally, this invention relates to neuroprotection methods, particularly by effect on hypoxia, lipid peroxidation and glucocorticoid excitotoxicity, hydrocortisol reduced production in the brain and other tissues, including the action on enzyme of conversion of cortisone→cortylol-11β-HSD1, or GRs-, or GITR-receptor suppression, blocking of glucocorticosteroid tissue activity indirectly through other targets, but without impairment of steroid hemostasis, compounds of the Formula I or pharmaceutically acceptable salts thereof of the Formula II.

Additionally, this invention relates to methods for 11β-HSD1activity inhibition comprising the interaction of 11β-HSD1 with compounds of the Formula I or pharmaceutically acceptable salts thereof of the Formula II.

Additionally, this invention relates to methods for inhibiting conversion of cortisone into cortisol (or 11 dehydrocorticosterone into 11β-corticosterone in rodents and other mammals) in the cells of human and animal tissues, comprising the interaction of cells with compounds of the Formula I or pharmaceutically acceptable salts thereof of the Formula II.

Additionally, this invention relates to methods of cortisol synthesis inhibition in human and animals cells, comprising the interaction of cells with compounds of the Formula I or pharmaceutically acceptable salts thereof of the Formula II.

Additionally, this invention relates to methods for treating various diseases, including any of the following diseases or any combination of two or more of the following diseases: as part of non-differentiated stroke therapy (without final verification of its subtypes) in different CVA periods, in recovery period after stroke and traumatic brain injury, in patients with chronic cerebrovascular pathology (including on the background of diabetes mellitus), for adjuvant therapy of Alzheimer's disease and encephalopathies of various origins (circulatory, alcohol, infectious-toxic), diabetes mellitus, for adjuvant therapy of retina degenerative eye diseases, as part of complex treatment of metabolic syndrome (adiposity, patients with Cushing syndrome, metabolic Riven syndrome (also known as X syndrome or syndrome of insulin resistance) and other diseases, insulin resistance, hyperglycemia, hypertension, hyperlipidemia, cognitive impairment; depression, dementia, glaucoma, heart disease; osteoporosis; inflammation; metabolic syndrome; excess of androgenic hormones or polycystic ovary syndrome (PCOS), comprising the administration to a patient of therapeutically effective amount of compounds of the Formula I or pharmaceutically acceptable salts thereof of the Formula II.

Additionally, this invention relates to the compound of the Formula I or pharmaceutically acceptable salts thereof of the Formula II for use in treatment of animals.

Additionally, this invention relates to the use of compounds of the Formula I or pharmaceutically acceptable salts thereof of the Formula II for making of a drug for use in therapy of above disease states.

This invention provides new pharmacological agents—spirocyclic compounds of 2-oxindole derivatives, containing the spiro[indolo-3,1′-pyrrolo[3,4-c]-pyrrol] core and remainders of biogenic sulphur-containing aminoacids (methionine, ethionine, cysteine and cysteine alkyl-derivatives), having the formula I:

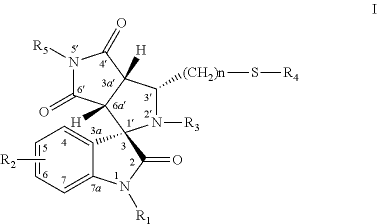

or pharmaceutically accepted salts thereof of the Formula II:

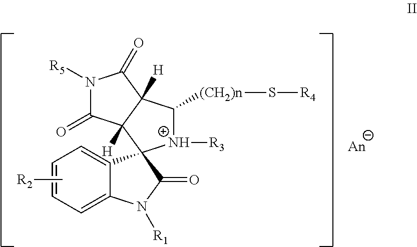

wherein

### R₁ is H, Me-, Et-, Alyl-, -Bn;

R₂ is H, 5-Me, 5-F, 5-Br, -5-OCF₃, 5-NO₂;

### R₃ is H or —N═O;

R₄ is residuals of biogenic sulphur-containing aminoacids (methionine (n=2, R₄=Me), or ethionine (n=2, R₄=Et), or cysteine (n=1, R₄=H) or cysteine alkyl-derivatives, wherein R₄=Bn or —CH₂CO₂Et, aσo Alyl-);
R₅ is H or remainders of Ar, wherein Ar is p-Tolyl, m-Tolyl, 2-(HO)Ph-, 3-(HO)Ph-, 4-Br-Ph-;
4-NO₂-Ph-; 2-NO₂-Ph-; 2-Br-Ph-; 4-(HOOC)Ph-;
and

An⁻ is selected from the group consisting of chloride, bromide, iodide, succinate, hemisuccinate, L-aspartate, tartrate or hydrotartrate, nicotinate, L-ascorbate, maleate or hydromaleate, fumarate, hydrofumarate, citrates, L-lactate, L-malate, phosphate, sulphate, benzoate, acetate, pivolate, glutarate, glutamate, asparaginate. Examples of pharmaceutically acceptable salts include, but are not limited to, salts of mineral or organic acids with basic residues such as amines; alkali metal salts or organic salts of acidic residues such as carboxylic acids; and the like. The pharmaceutically acceptable salts of present invention include conventional non-toxic salts of original compounds, prepared, for example, from non-toxic inorganic or organic acids. The pharmaceutically acceptable salts of present invention can be synthesized from the original compounds, which are bases, with conventional chemical methods. Typically, these salts can be prepared by reaction of free base form of these compounds with stoichiometric amount of corresponding acid in water or in organic solvent or mixtures thereof; usually non-aqueous environment of ethanol, isopropanol, or acetonitrile are preferred. The list of corresponding salts can be found in Remington's Pharmaceutical Sciences, 17th ed., Mack Publishing Company, Easton, Pa., 1985, p. 1418 and J. Pharm. Sci., 66, 2 (1977), each of which is incorporated herein in its entirety by reference.

The term “pharmaceutically acceptable” is used herein in relation to such compounds, materials, compositions and/or dosage forms that are safe and effective within careful medical judgment, suitable for use in contact with the tissues of humans and animals without exceeding the rate of toxicity, irritation, allergic reactions or other problems or complications with relatively acceptable risk/expected benefit.

All compounds and pharmaceutically acceptable salts thereof can be prepared in different solid forms, including hydrated or solvated forms. In some embodiments of the invention a solid is crystalline form. In particular, the process for preparation consists in environmentally acceptable one-stage enantioselective condensation of corresponding pyrrol-2,5-diones with 1H-indole-2,3-diones and biogenic sulfur-containing amino acids in polar medium, including methyl or isopropyl, or ethanol or acetonitrile mixed with water at a ratio of 2:1 to 10:1. The most appropriate ratio of alcohol:water is 3:1 ratio.

A process for preparation of salts of the compounds of general Formula I consists in dissolution of suitable base of these compounds in ethanol or a mixture of ethanol and water, or butanol, and adding aqueous or alcoholic solution of corresponding organic or inorganic acid, followed by evaporation in vacuum.

The process for preparation, purification and analysis of various solid forms are standard in the art and include, for example, X-ray powder diffraction, differential scanning colorimetry, thermogravimetric analysis, dynamic vapor sorption, FT-IR, Raman methods, NMR, titration by Karl-Fischer etc.

In some embodiments of the invention, compounds of present invention and salts thereof are almost completely isolated. By “almost completely isolated” we mean that the compounds at least partially or almost completely separated from environment in which there were formed or detected. Partial isolation may include, for example, composition enriched with compounds of present invention. Almost complete isolation may include composition comprising at least 50%, at least 60%, at least 70%, at least 80%, at least 90%, at least 95%, at least 97% or, at least 99% by weight of corresponding compound of present invention or salt thereof. The methods for compounds and salts isolation are standard in the art.

Compounds of the present invention are at nanomolar concentrations already and can cause neuroprotective effect, including via reducing hydrocortisol concentration in brains by action on the enzyme 11β-HSD1 of cortisone→cortylol conversion or inhibition of GRs-, or GITR-receptor or other target, but without interfering with steroid hemostasis in HPA. Compounds, salts thereof, and processes described in this invention, are benefit in reduction the hydrocortisone concentration. It is understood that the term “reducing the concentration of hydrocortisone (corticosterone)” refers to the ability to reduce the activity of the corresponding enzyme or receptor. Additionally, this invention relates to methods for inhibiting the conversion of cortisone to cortisol in a cell or inhibiting the synthesis of cortisol in a cell, wherein the conversion or synthesis of cortisol is mediated at least in part by 11β-HSD1 activity.

Additionally, this invention relates to methods for inhibiting conversion of cortisone to cortisol in a cell or inhibiting the synthesis of cortisol in a cell, wherein the conversion or synthesis of cortisol is mediated at least in part by 11β-HSD1 activity. Methods for measuring the rate of conversion of cortisone to cortisol and vice versa, as well as methods for measuring the concentrations of cortisone and cortisol in cells are standard in the art.

Additionally, this invention relates to methods for improving insulin sensitivity of cells by elimination of contrinsulinic glucocorticosteroid action at interaction of cells with compounds presented herein. The methods for measuring insulin sensitivity are standard in the art.

Additionally, this invention relates to methods for treating a disease associated with activity or expression, including abnormal activity and increased expression, of 11β-HSD1, in the individual by administrating to him/her a therapeutically effective amount or dose of corresponding compound of present invention or pharmaceutically acceptable salt thereof, or pharmaceutical composition based thereof. Examples of diseases can include any disease, disorder or condition that is directly or indirectly associated with the expression or activity of 11β-HSD1. Diseases associated with 11β-HSD1 can also include any disease, disorder or condition, which can be prevented, alleviated or treated by modulation of specified enzyme activity.

Compounds of the Formula can act as enzyme 11β-HSD1inhibitors.

Claimed compounds I and II reliably contribute to the normalization of cortisol in ischemic stroke model, indicating that they have a positive effect on modulating the formation of steroid excitotoxicity. The administration of compounds of present invention reduces only elevated cortisol level, and its titer does not differ from saline even during the course of therapy, which proves the lack of action on the hormonal HPA axis.

Examples of diseases associated with 11β-HSD1 include adiposity, diabetes, glucose intolerance, insulin resistance, hyperglycemia, hypertension, hyperlipidemia, cognitive disorders, dementia, stroke, depression (e.g., psychotic depression), glaucoma, heart disease, osteoporosis and inflammation. Additional examples of diseases associated with 11β-HSD1 include metabolic syndrome, diabetes type 2, excess of androgenic hormones (hirsutism, menstrual disorder, hyperandrogenism) and polycystic ovary syndrome (PCOS).

The term “cell” which is used herein refers to cells that exist in vitro, ex vivo or in vivo. In some embodiments of the invention ex vivo cell can be part of tissue sample obtained from an organism such as mammal. In still other embodiments of the invention in vitro cell can be a cell in cell culture. In some other embodiments of the invention in vivo cell is a cell that locates in a body such as a mammal, and is an adipose cell, pancreatic cell, hepatocyte, neuron or cell of the eye.

As used herein, the term “interaction” refers to the convergence of the above agents—compounds, enzymes, etc. in cells in vitro system or in vivo system. For example, the “interaction” of the enzyme 11β-HSD1 with the compounds of present invention comprises the administering of said compound to individual or patient, such as a human with 11β-HSD1, and for example, the administration of the compound in a sample containing a cellular or purified preparation or 11β-HSD1 enzyme.

As used herein, the term “individual” or “patient”, which are interchangeable, refers to any animal, including mammals, preferably mice, rats, and other rodents, rabbits, dogs, cats, pigs, cattle, sheep, horses or primates, and most preferably humans.

As used herein, the term “treat” or “treatment” refers to one or more of: (1) prevention of disease, for example, prevention of disease, condition or disorder in an individual prone to these diseases, condition or disorder but not yet felt or reveal their pathology or symptomatology; (2) termination of the disease; for example, termination of the disease, condition or disorder in an individual who felt or detects pathology or symptomatology of said disease, condition or disorder; and (3) alleviation of disease; for example, alleviation of disease, condition or disorder in an individual who felt or detects pathology or symptomatology of said disease, condition or disorder (i.e., reversal of pathology and/or symptomatology) such as reducing disease severity.

In use of compounds of present invention as drugs they may be administered in the form of pharmaceutical compositions which are combination of compounds of present invention and at least one pharmaceutically acceptable carrier.

These compounds can be prepared by processes well known in the pharmacy, and can be administered in various ways, depending on treatment needed—topical or systemic, and on disease requiring treatment.

The administration can be topical (including eye tissue, mucous membranes, including intranasal, vaginal and rectal delivery), pulmonary (e.g. by inhalation or insufflations of powders or aerosols, including aerosol products, intrathecal, intranasal, epidermal and transdermal), oral or parenteral.

Method for administration in ophthalmology may include: local administration (eye drops) underconjuctival, periocular injectable solution or solution for administration into vitreous body or administration with balloon catheter or ophthalmic film administration which are administered into conjunctival sac.

Parenteral administration comprises intravenous, intraarterial, subcutaneous, intraperitoneal or intramuscular injection or infusion; or intracranial, e.g., intrathecal or intraventricular administration. Parenteral administration may be in the form of single bolus dose, or may be made, for example, by means of continuous perfusion pump.

Pharmaceutical compositions and compositions for topical administration may comprise transdermal patches, ointments, lotions, creams, gels, drops, suppositories, sprays, liquids and powders. Generally accepted pharmaceutical carriers, aqueous, powder or oily bases, thickeners and similar excipients may be necessary and desirable.

This invention also provides pharmaceutical compositions comprising as an active ingredient one or more of the above compounds of general Formula I in combination with one or more pharmaceutically acceptable carriers. At preparation of compositions of present invention the active ingredient is usually mixed with adjuvants, diluted with adjuvants or arranged in appropriate carrier in the form of, for example, capsules, sachets, paper or other reservoir. When the excipient is a diluent, it may be solid, semi-solid or liquid material which acts as a solvent, carrier or medium for the active ingredient. Thus, the compounds may be in the form of tablets, pills, powders, lozenges, sachets, starch capsules, elixirs, suspensions, emulsions, solution s, syrups, aerosols (as a solid or in liquid medium), ointments containing, for example, up to 10% by weight of the active compound, soft and hard gelatin capsules, suppositories, sterile injectable solution s and packaged in a sterile powders.

Compounds of present invention can be grinded using known methods such as wet grinding, to obtain particle sizes suitable for obtaining tablets and other types of compositions. Finely ground (nanoparticles) preparations of compounds of present invention can be prepared by methods known in the art, for example, se International patent application No. WO 2002/000196.

Some examples of suitable excipients include lactose, dextrose, sucrose, sorbitol, mannitol, starch, gum acacia, calcium phosphate, alginates, tragacanth, gelatin, calcium silicate, microcrystalline cellulose, polyvinylpyrrolidone, cellulose, water, syrup and methylcellulose. Compositions may further comprise lubricants such as talc, magnesium stearate and mineral oil; wetting agents; emulsifying and suspending agents; preservatives such as methyl and propylhydroxybenzoate; sweeteners and flavoring agents. The compounds of present invention can be formulated so as to provide rapid, sustained or delayed release of active ingredient after administration to a patient using methods known in the art.

The compounds can be formed in a single dosage form, and each dose contains from about 5 to about 100 mg, more preferably from about 10 to about 50 mg of the active ingredient. The term “unit dosage form” refers to physically discrete unit forms suitable as a single dose to humans and other mammals, and every single form contains predetermined amount of active material calculated to provide desired therapeutic effect together with appropriate pharmaceutical excipients.

The active compounds can be effective over a wide dose range, and usually administered in a pharmaceutically effective amount. However, it is common that therapeutically effective amount of compounds can be adjusted by the attending physician considering the surrounding circumstances, including the state that requires treatment, route of administration of the therapeutic agent, specific compounds administered,] age, weight and response of particular patient, severity of symptoms in a patient etc.

To prepare solid compositions such as tablets, the main active ingredient is mixed with pharmaceutical excipients to produce solid precursor compound containing homogeneous mixture of compounds of present invention. When referring to data of precursor compositions as homogeneous, an active ingredient is usually evenly distributed in the composition so that the composition can be easily divided into equal effective unit dosage forms such as tablets, pills and capsules. These solid precursor compositions then could be divided into single dosage forms of type described above containing from, for example, 0.1 to about 500 mg of active ingredient of present invention. The tablets or pills of present invention may be coated or otherwise mixed to prepare medical form to have long-acting effect. For example, a tablet or pill can contain internal dose and external dose component, and the latter is the coating for the first one. Two components can be divided with enteric layer that serves to resist disintegration in the stomach and allows the inner component to pass intact into the duodenum or used for slowing the release. One can use a plurality of materials for these enteric layers or coatings, and these materials comprise a number of polymeric acids and mixtures of polymeric acids with such materials as shellac, cetyl alcohol and cellulose derivatives, in particular hydroxypropylmethylcellulose or ethylcellulose etc.

Liquid forms, in which compounds of present invention can be administered for oral administration or administration by injection comprise aqueous solution s, appropriate flavored syrups, aqueous or oily suspensions, and flavored emulsions with nutrient oils such as cottonseed oil, sesame oil, coconut oil or peanut oil, as well as elixirs and similar pharmaceutical solvents.

The compounds for inhalation or insufflations comprise solutions and suspensions in pharmaceutically acceptable aqueous or organic solvents, or mixtures thereof, and powders. Liquid or solid compounds may contain suitable pharmaceutically acceptable excipients as described above. In some embodiments, the compounds are administered orally or by nasal respiratory route for topical or systemic action. The compounds can be sprayed using inert gases. Solution s that are sprayed can be inhaled directly from the spraying device or spraying device can be connected to facial masks or breathing device with positive interspersed pressure.

The compositions as solution, suspension or powder can be administered orally or nasally with a device that delivers said composition by appropriate method.

Amount of a compound administered to a patient will depend on the form of administered compounds or composition, purpose of administration, such as the prevention or treatment of a patient, route of administration and so on. For therapeutic purposes the compounds can be administered to a patient already suffering from a disease in amount sufficient to treat or at least partially terminate the symptoms of the disease and its complications. Effective doses will depend on the state of disease that needs to be treated and the severity of disease, age, weight and general state of the patient etc.

The compounds administered to a patient may be in the form of pharmaceutical compositions described above. These compositions can be sterilized with accepted methods of sterilization or they can be sterilized by sterilizing filtration.

Aqueous solutions can be packaged for use as is or freeze-dried and lyophilized drug is mixed with sterile aqueous carrier prior to administration. pH of these drugs usually will be from 3 to 11, more preferably from 5 to 9 and most preferably from 7 to 8. It is clear that the use of certain substances from above excipients, carriers, or stabilizers will result in the formation of pharmaceutical salts.

Therapeutic doses of compounds of present invention may vary depending, for example, on the particular use for which the treatment is carried out, route of administration, and health state of a patient, and physician decision. The proportion or concentration of compounds of present invention in pharmaceutical composition may vary depending on number of factors including dosage, chemical characteristics (e.g., hydrophobicity), and administration way. For example, compounds of present invention can be prepared in aqueous physiological buffer solution containing from about 0.1 to about 10% wt./vol. compounds for parenteral administration. Some ranges of the standard dose are from about 1 mg/kg to about 1 g/kg body weight per day. In some embodiments, the dose range is from about 0.01 mg/kg to about 100 mg/kg body weight per day. The dose likely depends on such variables as a type and extent of progression of the disease or disorder, overall health state of the individual patient, relative biological activity of selected compounds, excipient composition and its way of administration. Effective doses can be extrapolated based on dose-effect curves obtained for test systems in vitro or animal model.

Compounds of present invention can also be used in combination with one or more additional active pharmaceutical ingredients, which can comprise any pharmaceutical agent such as antiviral agents, vaccines, antibiotics, agents that enhance immunity, immunosuppressive, anti-inflammatory agents, analgesics and drugs for the treatment of stroke, heart attack, diabetes and adiposity, hyperglycemia, hypertension, hyperlipidemia and the like. Agents for treatment of metabolic disorders, with which compounds of present invention can be mixed, including but not limited to, amilin analogues, incretine mimetics, inhibitors of dypeptydylpeptydase-IV, which is an enzyme that breaks down incretine, receptor agonists that activate peroxisome proliferator (PPAR)-a and PPAR-g, and inhibitor of CB 1 cannabinoid receptor.

This invention will be described in more detail using specific examples. The following examples are for illustrative purposes and should not be considered as limiting this invention in any way. Those skilled in the art should realize that the set of non-critical parameters that can be changed or modified, will give essentially the same results.

### EXAMPLES

All compounds were purified by column or flash chromatography or reverse phase liquid chromatography using Waters FractionLynx LC-MS system with fractionation by weight. Column: Waters XBridge C18 5 mm, 19×100 mm; mobile phase A: 0.15% NH₄OH in water and mobile phase B: 0.15% NH₄OH in acetonitrile; flow rate was 30 ml/min., separating gradient for each selected compound, using 15 Compound Specific Method Optimizathion protocol, as described in the literature [“Preparative LC-MS Purification: Improved Compound Specific Method Optimizathion”, K. Blom, B. Glass, R. Sparks, A. Combs, J. Combi. Chem., 2004, 6, 874-883].

Then the selected product is usually subjected to analytical LC/MS to verify the purity of the following conditions: Instrument; Agilent 1100 series, LC/MSD, column: Waters Sunfire™ C18 5 micron, 20 2.1×5.0 mm, buffers, mobile phase A: 0.025% TFA in water and mobile phase B: 0.025% TFA in acetonitrile; gradient 2%-80% buffer B for 3 min at flow rate of 1.5 ml/min.

### Example 1

### 5′-(4-Methylphenyl)-3′-[2-(methylthio)ethyl]-3a′,6a′-dihydro-2′H-spiro[indole-3,1′-pyrrolo[3,4-c]pyrrol]-2,4′,6′(1H, 3′H,5′H)-trione

### 5′-(4-Methylphenyl)-3′-[2-(methylthio)ethyl]-3a′,6a′-dihydro-2′H-spiro[indole-3,1′-pyrrolo[3,4-c]pyrrol]-2,4′,6′(1H,3′H,5′H)-trione (SI-86)

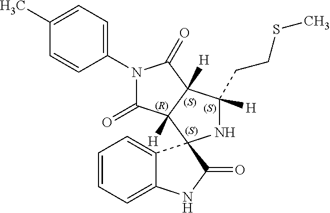

### Stage 1: 1-p-Tolyl-pyrrol-2,5-dione (1-p-Tolyl-pyrrol-2,5-dione)

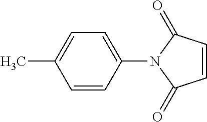

Maleic anhydride (98.1 g, 1.0 mol) and p-tolyidine (137.1 g, 1.0 mol) were dissolved in N,N-dimethylformamide (DMF, 320 ml), then the mixture was mixed at room temperature for 5 hours in nitrogen atmosphere. Prepared solution was poured into large amount of water to precipitate raw p-tolylmaleamic acid (N-(4-methylphenyl)maleamic acid, p-TMA). Raw p-tolylmalic acid was filtered, dried and then recrystallized three times with the mixture of water producing purified product (97%). Melting T. 160-162° C.

The mixture of p-TMA (43.5 g, 0.2 mol), acetic anhydride (100 ml) and (2.5 g) sodium acetate mixed at 55-60° C. for 2 hours. The reaction mixture was poured into large amount of ice resulting in raw 1-p-tolyl-pyrrol-2,5-dione as oily precipitate solidifying in amorphous mass at mixing. After that the precipitate is decanted and raw 1-p-tolyl-pyrrol-2,5-dione was dissolved in minimal amount of ethanol, left at 0-+10° C. till precipitate formation, which was filtered, washed with water, dried and recrystallized three times from ethanol. Yield 85%. T_{melt.}=144-146° C. Yellow crystal powder. LC/MS m/e 188 (m+H)⁺. NMR ¹H, δ, m.f (TMS, DMSO-d₆): 8.03-7.49 (2d, J=8.24 Hz, 4H, Ph); 7.22 (s, 2H, —CO—CH═CH—CO—), 2.33 (3H, s, PhCH₃). ¹³C NMR (CDCl₃) δ 21.1, 126.0, 128.5, 129.8, 134.2, 138.1, 169.7. IR (CHCl₃, cm⁻¹) 1708, 1390.

### Stage 2: 5′-(4-Methylphenyl)-3′-[2-(methylthio)ethyl]-3a′,6a′-dihydro-2′H-spiro[indole-3,1′-pyrrolo [3,4-c]pyrrol]-2,4′,6′(1H,3′H,5′H)-trione (SI-86)

The mixture (1 mol, 147 g) of isatine, (1 mol, 147 g) L-methionine (≧99.0% (NT) (Fluka) and (1 mol, 187 g) 1-p-tolyl-pyrrol-2,5-dione, in 40 ml of mixture propan-2-ol:water (3:1) is boiled for 2,5-3 hours, reaction is controlled by TLC and color changes of reaction mixture (from red to straw). The solution was cooled, precipitate was filtered, washed with propan-2-ol and crystallized from n-butanol calculated for 70 ml per 1 g. Yield 366 g, 87%. White crystal powder, Melting T. 184-186° C. (n-BuOH), absorption maximum λₘₐₓ 225 nm (Igε˜3.703) in methanol. LC/MS m/e 421 (m+H)⁺. NMR ¹H, δ, m.f (TMS, DMSO-D₆): 10.39 (1H, s, 1H, NH), 7.26-7.38 (2H, m, Ar), 7.11-7.23 (3H, m, Ar), 6.74-7.02 (3H, m, Ar), 4.26 (1H, t, J=7 Hz, 3′-CH), 3.85 (1H, d, J=7 Hz, 2′-NH), 3.61 (1H, t, J=8 Hz, 3a′-CH), 3.41 (1H, d, J=8 Hz, 6a′-CH), 3.27-3.33 (2H, m, CH₂CH₂SCH₃), 2.54-2.68 (2H, m, CH₂CH₂SCH₃), 2.34 (3H, s, PhCH₃), 1-94-2.17 (3H, m, CH₂CH₂SCH₃), 1-72 (1H, dd, J=14 and 7 Hz). Found, %: C. 65.53; H. 5.50; N. 9.95; S. 7.62. C₂₃H₂₃N₃O₃S. Calculated. %: C. 65.54; H. 5.50; N. 9.97; S. 7.61.

### Example 2

### 5′-(4-Methylphenyl)-3′-[2-(ethylthio)ethyl]-3a′,6a′-dihydro-2′H-spiro[indole-3, 1′-pyrrolo[3,4-c]pyrrol]-2,4′,6′(1H,3H′,5H′)-trione (SI-34)

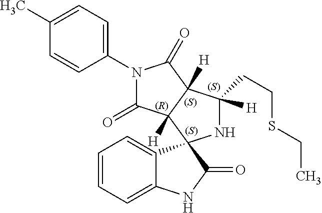

This compound prepared, using techniques, similar to those, described for synthesis of the Example 1, stages 1 i 2, and instead of L-methionine L-ethionine was used. Yield 88%. White crystal powder, Melting T. 209-210° C. (with decomposition, n-BuOH), absorption maximum λₘₐₓ 291 nm (Igε˜0.618) in methanol. LC/MS m/e 435 (m+H)⁺. NMR ¹H, δ, m.f (TMS, DMSO-D₆): 10.39 (1H, s, 1H, NH), 7.18 (4-Ar, 1H, T; J=7.6) 6.98 (4-Ar, 1H, [symbol] J=7.9 Hz), 6.88 (5-Ar, 1H, T J=7.9), 6.80 (7-Ar, 1H, [symbol] J=7.9), 4.31-4.24 (3′-CH, 1H, M,), 3.86 (1H, d, J=6.7 Hz, 2′-NH), 3.62 (3′a-CH 1H, T J=7.6), 3.41 (1H, d, J=8 Hz, 6a′-CH), 2.13-2.04; 1.78-1.69 (2H, M, 3′-CHaHb-CH₂S), 2.72-2.62 (2H, M, 3′-CHaHb-CH₂S), 2.54-2.50 (3′-SCH₂Me, 2H, M), (3′-SCH₂Me J=7.3), 2.35 (3H, s, PhCH₃), 1.18 (3H, [symbol] 3′-SCH₂Me T J=7.3). Found, %: C, 66.20; H, 5.79; N, 9.67; S 7.37. C₂₄H₂₅N₃O₃S. Calculated, %: C, 66.18; H, 5.79; N, 9.65; S 7.36.

### Example 3

### 5-Bromo-5′-(4-methylphenyl)-3′-[2-(ethylthio)ethyl]-3a′,6a′-dihydro-2′H-spiro[indole-3,1′-pyrrolo[3,4-c]pyrrolo]-2,4′,6′(1H,3′H,5′H)-trione (SI-76 5t)

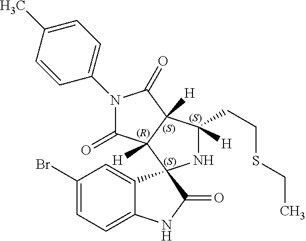

This compound prepared, using techniques, similar to those, described for synthesis of the Example 1, stages 1 i 2, and instead of L-methionine and isatin L-ethionine and 5-bromoisatin were used, respectively. Yield 90%. Creamy crystal powder, Melting T. 243-245° C. (n-BuOH). LC/MS m/e 514 (m+H)⁺. Spectrum NMR ¹H, δ, m.f (J, Hz): 1.17 (T, 3H, CH₂CH₂SCH₂CH₃, J=7.5), 1.66-2.12 (m, 2H, CH₂CH₂SCH₂CH₃), 2.34 (c, 3H, PhCH₃), 2.50-2.73 (m, 4H, CH₂CH₂SCH₂CH₃), 3.46 (d, 1H, 6a′-CH, J=7.7), 3.64 (T, 1H, 3a′-CH, J=7.7), 3.98 (d, 1H, 2′-NH, J=7.0), 4.12-4.23 (m, 1H, 3′-CH), 6.76 (d, 1H, 7-CH, J=8.4), 7.12 (d, 1H, 4-CH, J=1.5), 7.17 (d, 2,6-CH (PhMe), J=8.1), 7.31 (d, 2H, 3,5-CH (PhMe), J=8.1), 7.37 (dd, 1H, 6-CH, Jₘₑₜₐ=1.5, Jₒᵣₜₕₒ=8.4), 10.57 (c, 1H, NH). ¹³C NMR (DMSO-d₆) δ: 180.1, 176.0, 174.6, 142.0, 138.5, 132.3, 130.4, 130.3, 129.9, 129.5, 127.2, 113.2, 111.8, 68.8, 58.1, 53.2, 49.3, 40.6, 40.5, 40.4, 40.3, 40.3, 40.2, 40.1, 40.0, 39.9, 39.8, 39.7, 39.5, 31.8, 29.1, 25.4, 21.2, 15.3. Found, %: C, 56.02; H. 4.69; N. 8.16; S. 6.22. C₂₄H₂₄BrN₃O₃S. Calculated. %: C. 56.03; H. 4.70; N. 8.17; S. 6.23.

### Example 4

### 5-Bromo-5′-(4-methylphenyl)-3′-[2-(methylthio)ethyl]-3a′,6a′-dihydro-2′H-spiro[indole-3,1-pyrrolo [3,4-c]pyrrol]-2,4′,6′(1H,3′H,5′H)-trione

### 5-Bromo-5′-(4-methylphenyl)-3′-[2-(methylthio)ethyl]-3a′,6a′-dihydro-2′H-spiro[indole-3,1′-pyrrolo[3,4-c]pyrrol]-2,4′,6′(1H,3′H,5′H)-trione (SI-87 6v)

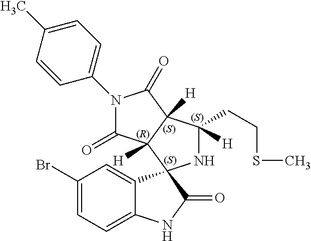

This compound prepared, using techniques, similar to those, described for synthesis of the Example 1, stages 1 and 2, and instead of isatine 5-bromoisatin was used respectively. Yield 90%. Creamy crystal powder, Melting T. 242-244° C. (n-BuOH). LC/MS m/e 500 (m+H)⁺. ¹H NMR (200 MHz, DMSO-d₆, TMS) δ: 10.56 (c, 1H, NH), 7.42-7.08 (m, 6H, Ar) 6.83-6.73 (m, 1H, Ar), 4.33-4.16 (m, 1H, NH), 3.93-3.83 (m, 1H, CH), 3.65-3.46 (m, 2H, CH), 2.34 (c, 3H, CH₃), 1.25 (d, 3H, CH₃). Found, %: C, 55.23; H, 4.50; N, 8.40. C₂₃H₂₂BrN₃O₃S, %. Calculated, %: C, 55.20; H, 4.43; N, 8.40.

### Example 5

### 1-Methyl-5′-(4-Methylphenyl)-3′-[2-(methylthio)ethyl]-3a′,6a′-dihydro-2′H-spiro[indole-3,1′-pyrrolo[3,4-c]pyrrol]-2,4′,6′(1H,3′H,5′H)-trione (SI-176N)

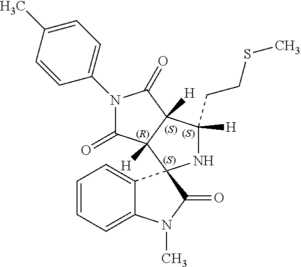

This compound prepared, using techniques, similar to those, described for synthesis of the Example 1, stages 1 and 2, and instead of isatine 1-methyl-isatin was used respectively. Yield 95%. White crystal powder, Melting T. 257-259° C. (n-BuOH). LC/MS m/e 435 (m+H)⁺. ¹H NMR (400 MHz, DMSO-d₆, TMS): δ 7.33 (d, J=7.9 Hz, 3H), 7.22 (d, J=8.3 Hz, 2H), 6.91-7.08 (m, 3H), 4.31 (br. s., 1H), 3.84-3.95 (m, 1H), 3.66 (t, J=7.5 Hz, 1H), 3.45 (d, J=7.9 Hz, 1H), 3.32 (br. s., 2H), 3.13 (s, 3H), 2.58-2.73 (m, 2H), 2.51 (br. s., 1H), 2.37 (br. s., 3H), 2.01-2.13 (m, 4H), 1.69-1.83 (m, 1H). Found, %: C, 66.19; H, 5.80; N, 9.66; S, 7.37. C₂₄H₂₅N₃O₃S. Calculated, %: C, 66.18; H, 5.79; N, 9.65; S, 7.36.

### Example 6

### 1-(4-Chlorobenzyl)-3′-[2-(ethylthio)ethyl]-5′-(4-methylphenyl)-3a′,6a′dihydro-2′H-spiro[indole-3,1′-pyrrolo[3,4-c]pyrrol]-2,4′,6′(1H,3′H,5′H)′trione

### 1-(4-Chloro-benzyl)-3′-[2-(ethylthio)ethyl]-5′-(4-methylphenyl)-3a′,6a′-dihydro-2′H-spiro[indole-3,1′-pyrrolo[3,4-c]pyrrol]-2,4′,6′(1H,3′H,5′H)-trione (SI-108 6×)

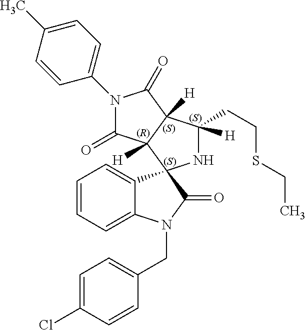

This compound prepared, using techniques, similar to those, described for synthesis of the Example 1, stages 1 and 2, and instead of L-methionine and isatin L-ethionine and 1-chlorobenzyl-isatin were used respectively. Yield: 63%. White amorphous powder with melting T. 158-160° C. (n-BuOH). LC/MS m/e 559 (m+H)⁺. ¹H NMR (200 MHz, DMSO-d₆, TMS): δ 7.44-6.83 (m, 12H, Ar), 4.85 (d, 2H, CH₂), 4.42-4.23 (m, 1H, NH), 3.98 (d, 1H, CH), 3.76-3.60 (m, 2H, CH), 3.50 (d, 3H, CH, CH₂), 2.74-2.53 (d, 2H, CH₂), 2.34 (s, 3H, CH₃), 2.16-1.64 (m, 4H, CH₂), 1.17 (t, 3H, CH₃). Calculated, %: C, 66.48; H, 5.40; N, 7.50. C₃₁H₃₀CIN₃O₃S. Found, %: C, 66.50; H, 5.43; N, 7.49.

### Example 7

### 5-Bromo-3′-[2-(ethylthio)ethyl]-3a′,6a′-dihydro-2′H-spiro[indolo-3,1′-pyrrolo[3,4-c]pyrrol]-2,4′,6′(1H,3′H,5′H)-trione

### 5-Bromo-3′-[2-(ethylthio)ethyl]-3a′,6a′-dihydro-2′H-spiro[indole-3, 1′-pyrrolo[3,4-c]pyrrol]-2,4′,6′(1H,3′H, 5′H)-trione (SI-81 7c)

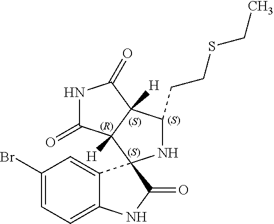

This compound prepared, using techniques, similar to those, described for synthesis of the Example 1, stages 1 and 2, and instead of 1-p-tolyl-pyrrol-2,5-dione L-methionine and isatin, pyrrol-2,5-dione, L-ethionine and 5-bromo-isatin were used respectively. Yield: 75%. White crystal powder with melting temperature 228-230° C. (n-BuOH). LC/MS m/e 424 (m+H)⁺. ¹H NMR (200 MHz, DMSO-d₆, TMS) δ: 1.17 (T, J=7.32 Hz, 4H) 1.47-1.71 (m, 1H) 1.88-2.11 (m, 1H) 2.34-2.72 (m, 7H) 3.24 (d, J=7.69 Hz, 3H) 3.38-3.53 (m, 1H) 3.86 (d, J=5.86 Hz, 1H) 4.03-4.21 (m, 1H) 6.74 (d, J=8.42 Hz, 1H) 7.04 (c, 1H) 7.37 (d, J=8.06 Hz, 1H) 10.49 (c, 1H) 11.39 (c, 1H). ¹³C NMR (DMSO-d₆) δ: 180.3, 178.3, 176.8, 141.9, 132.1, 130.6, 129.3, 113.2, 111.6, 68.1, 57.3, 53.9, 49.8, 40.5, 40.3, 40.2, 40.0, 39.8, 39.7, 39.5, 32.0, 29.0, 25.4, 15.3. Calculated, %: C, 48.12; H, 4.28; N, 9.90. C₁₇H₁₈BrN₃O₃S. Found, %: C, 48.15; H, 4.30; N, 9.95.

### Example 8

### 1-Allyl-3′-[2-(methylthio)ethyl]-3a′,6a′-dihydro-2′H-spiro[indole-3,1′-pyrrolo[3,4-c]pyrrol]-2,4′,6′(1H,3′H,5′H)-trione

### 1-Allyl-3′-[2-(methylthio)ethyl]-3a′,6a′-dihydro-2′H-spiro[indole-3,1′-pyrrolo[3,4-c]pyrrol]-2,4′,6′(1H,3′H,5′H)-trione (SI-149 7d)

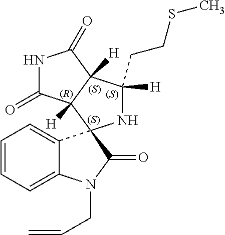

This compound prepared, using techniques, similar to those, described for synthesis of the Example 1, stages 1 and 2, and instead of 1-p-tolyl-pyrrol-2,5-dione and isatin, pyrrol-2,5-dione, and 1-allyl-isatin were used, respectively. Yield: 75%. White crystal powder with melting temperature 300° C. (with decomposition, n-BuOH). LC/MS m/e 371 (m+H)⁺. ¹H NMR (200 MHz, DMSO-d₆, TMS): δ 1.18 (d, J=6.59 Hz, 4H) 3.10-3.27 (m, 2H) 3.35-3.46 (m, 2H) 3.70 (d, J=5.49 Hz, 1H) 4.11-4.35 (m, 3H) 5.00-5.28 (m, 2H) 5.68-5.94 (m, 1H) 6.81-7.10 (m, 3H) 7.25 (d, J=7.32, 1.83 Hz, 1H) 11.29 (br. s, 1H). Calculated, %: C, 61.44; H, 5.70; N, 11.31. C₁₉H₂₁N₃O₃S. Found, %: C, 61.48; H, 5.77; N, 11.36.

### Example 9

### 1-Allyl-3′-(mercaptomethylen)-3a′,6a′-dihydro-2′H-spiro[indole-3, 1′-pyrrolo[3,4-φipoπ]-2,4′,6′(1H,3′H, 5′H)-trione

### 1-AllyI-3′-[mercaptomethylen]-3a′,6a′-dihydro-2′H-spiro[indole-3,1′-pyrrolo[3,4-c]pyrrol]-2,4′,6′(1H,3′H,5′H)-trione (SI-123)

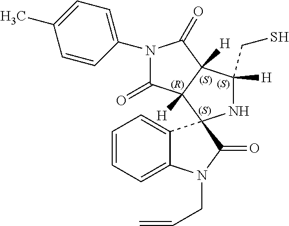

This compound prepared, using techniques, similar to those, described for synthesis of the Example 1, stages 1 and 2, and instead of L-methionine and isatin L-cysteine hydrochloride and 1-allyl-isatin were used respectively, maintained the reaction mixture at temperature not higher than 80-90° C. The reaction mixture was filtered, concentrated in vacuum and residual was purified by flash chromatography on silicagel column (eluating with ethylacetate:hexanes mixture (1:1)) to prepare end product. Yield: 52%. Slightly yellowish crystal powder with melting temperature 158° C. (with decomposition, n-BuOH). LC/MS m/e 371 (m+H)⁺. ¹H NMR (200 MHz, DMSO-d₆, TMS): δ 1.03 (br. s, 11H), 2.33 (br. s, 12H), 4.07-4.60 (m, 9H), 5.10-5.34 (m, 4H), 5.80 (br. s, 2H), 6.86-7.03 (m, 4H), 7.11 (m, J=7.32 Hz, 5H) 7.28 (m., 3H). Calculated, %: C, 66.49; H, 5.35; N, 9.69; S, 7.40. C₂₄H₂₃N₃O₃S. Found, %: C, 66.47; H, 5.36; N, 9.70; S, 7.42.

### Example 10

### 1-Methyl-3′-(mercaptomethylen)-3a′,6a′-dihydro-2′H-spiro[indole-3,1′-pyrrolo[3,4-c]pyrrol]-2,4′,6′(1H,3′H,5′H)-trione

### 1-Methyl-3′-(mercaptomethylen)-3a′,6a′-dihydro-2′H-spiro[indole-3,1′-pyrrolo[3,4-c]pyrrol]-2,4′,6′(1H,3′H,5′H)-trione (SI-124)

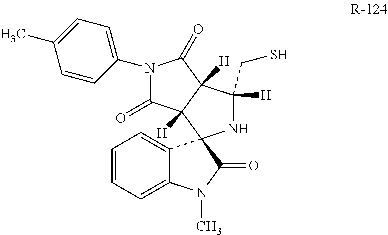 [SMILES: Cc1ccc(N2C(=O)[C@@H]3[C@H](CS)N[C@@]4(C(=O)N(C)c5ccccc54)[C@@H]3C2=O)cc1]

This compound prepared, using techniques, similar to those, described for synthesis of the Example 1, stages 1 and 2, and instead of L-methionine and isatin L-cysteine hydrochloride and 1-methyl-isatin were used, respectively, maintained the reaction mixture at temperature not higher than 80-90° C. The reaction mixture was filtered, concentrated in vacuum and residual was purified by flash chromatography on silicagel column (eluating with ethylacetate:hexanes mixture (1:1)) to prepare end product. Yield: 45%. Slightly yellowish crystal powder with melting temperature 167-169° C. (with decomposition, n-BuOH). LC/MS m/e 407. ¹H NMR (200 MHz, DMSO-d₆, TMS) δ: 1.05 (d, J=5.86 Hz, 2H) 2.36 (br. s, 4H) 2.51 (m., 4H) 3.14 (br. s, 3H) 3.78 (br. s, 2H) 4.18 (br. s, 2H) 4.56 (br. s, 2H) 7.15 (br. s, 3H) 7.30 (d, J=7.69 Hz, 5H). Calculated, %: C. 64.85; H. 5.19; N. 10.31; S. 7.87. C₂₂H₂₁N₃O₃S. Found. %: C. 64.86; H. 5.20; N. 10.32; S. 7.88.

### (00) Example 11

### 3′-(Mercaptomethylen)-3a′,6a′-dihydro-2′H-spiro[indole-3,1′-pyrrolo[3,4-c]pyrrol]-2,4′,6′(1H,3′H,5′H)-trione

### 3′-(Mercaptomethylen)-3a′,6a′-dihydro-2′H-spiro[indole-3,1′-pyrrolo[3,4-c]pyrrol]-2,4′,6′(1H,3′H,5′H)-trione (SI-121)

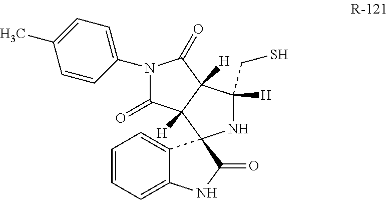 [SMILES: Cc1ccc(N2C(=O)[C@@H]3[C@H](CS)N[C@@]4(C(=O)Nc5ccccc54)[C@@H]3C2=O)cc1]

This compound prepared, using techniques, similar to those, described for synthesis of the Example 1, stages 1 and 2, and instead of L-methionine L-cysteine hydrochloride was used, maintained the reaction mixture at temperature not higher than 80-90° C. The reaction mixture was filtered, concentrated in vacuum and residual was purified by flash chromatography on silicagel column (eluating with ethylacetate:hexanes mixture (1:1)) to prepare end product. Yield: 43%. Slightly yellowish crystal powder with melting temperature 178° C. (with decomposition, n-BuOH). LC/MS m/e 393. ¹H NMR (200 MHz, DMSO-d₆, TMS): 2.33 (c, 3H) 3.26 (wide s, 3H) 3.67-3.87 (m, 1H) 4.07-4.24 (m, 1H) 4.51 (d, J=5.04 Hz, 1H) 6.74-6.99 (m, 1H) 6.99-7.39 (m, 8H) 10.55 (br. s, 1H). Calculated, %: C, 64.10; H. 4.87; N. 10.68; S. 8.15. C₂₁H₁₉N₃O₃S. Found. %: C. 64.08; H. 4.88; N. 10.69; S. 8.14.

### (01) Example 12

### 1-Allyl-3′-[2-(ethylthio)ethyl]-5′-(4-methylphenyl)-3a′,6a′-dihydro-2′H-spiro[indole-3,1′-pyrrolo[3,4-c]pyrrol]-2,4′,6′(1H,3′H,5′H)-trione

### 1-Allyl-3′-[2-(ethylthio)ethyl]-5′-(4-methylphenyl)-3a′,6a′-dihydro-2′H-spiro[indole-3,1′-pyrrolo[3,4-c]pyrrol]-2,4′6′(1H,3′H,5′H)-trione (SI-175)

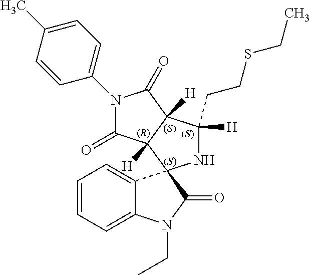

This compound prepared, using techniques, similar to those, described for synthesis of the Example 1, stages 1 and 2, and instead of L-methionine and isatin, L-ethionine and 1-allyl-isatin were used, respectively. Yield: 85%. White crystal powder with melting temperature 275-277° C. (with decomposition, n-BuOH). LC/MS m/e 475 (m+H)⁺. ¹H NMR (200 MHz, DMSO-d₆, TMS): 7.08-7.37 (5H, m), 6.86-7.06 (3H, m), 5.73-5.94 (1H, m), 5.10-5.30 (2H, m), 4.27 (3H, br. s.), 3.93 (1H, d, J=7 Hz), 3.57-3.72 (1H, m, M07), 3.41 (1H, d, J=8 Hz), 2.56-2.73 (2H, m), 2.34 (3H, s), 1.96-2.17 (2H, m), 1.61-1.82 (1H, m), 1.09-1.23 (3H, m). Calculated, %: C, 68.18; H. 6.15; N. 8.84; S. 6.74. C₂₇H₂₉N₃O₃S. Found. %: C. 68.19; H. 6.17; N. 8.85; S. 6.74.

### (02) Example 13

### 5-Fluoro-3′-[2-(ethylthio)ethyl]-5′-(4-methylphenyl)-3a′,6a′-dihydro-2′H-spiro[indolo-3,1′-pyrrolo[3,4-c]pyrrol]-2,4′,6′(1H,3′H,5′H)-trione

### 5-Fluoro-3′-[2-(ethylthio)ethyl]-5′-(4-methylphenyl)-3a′,6a′-dihydro-2′H-spiro[indole-3,1′-pyrrolo[3,4-c]pyrrol]-2,4′,6′(1H,3′H,5′H)-trione (SI-180F)

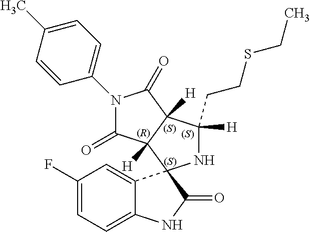

This compound prepared, using techniques, similar to those, described for synthesis of the Example 1, stages 1 and 2, and instead of L-methionine and isatin, L-ethionine and 5-fluoro-1H-indole-2,3-dione were used, respectively. Yield: 85%. White crystal powder with melting temperature 285-287° C. (with decomposition, n-BuOH). LC/MS m/e 453 (m+H)⁺. ¹H NMR (200 MHz, DMSO-d₆, TMS) δ: 10.46 (1H, s, NH), 6.90-7.38 (6H, m, Ar), 6.70-6.90 (2H, m, Ar), 4.24 (1H, t, J=7 Hz, 1H, 3′-CH), 3.94 (1H, d, J=7 Hz, 2′-NH), 3.62 (1H, t, J=8 Hz, 3a′-CH), 3.45 (1H, d, J=8 Hz, 6a′-CH), 2.55-2.72 (2H, m, CH₂CH₂SCH₂CH₃), 2.27-2.39 (4H, m, PhCH₃+CH₂CH₂SCH₂CH₃), 2.06 (1H, dq, J=14 and 7 Hz,), 1.57-1.80 (1H, c, CH₂CH₂SCH₂CH₃), 1.09-1.23 (3H, d, CH₂CH₂SCH₂CH₃). ¹³C NMR (DMSO-d₆) δ: 180.5, 135.1, 129.9, 129.8, 127.3, 127.1, 69.0, 57.9, 53.1, 49.1, 40.6, 40.5, 40.4, 40.3, 40.3, 40.2, 40.1, 40.0, 39.9, 39.8, 39.7, 39.5, 31.9, 29.1, 25.4, 21.2, 15.3 Calculated, %: C, 63.56; H. 5.33; N. 9.27; S. 7.07. C₂₄H₂₄FN₃O₃S. Found. %: C. 63.55; H. 5.34; N. 9.26; S. 7.06.

### (03) Example 14

### 5-Fluoro-3′-[2-(ethylthio)ethyl]-3a′,6a′-dihydro-2′H-spiro[indolo-3,1′-pyrrolo[3,4-c]pyrrol]-2,4′,6′(1H,3′H, 5′H)-trione

### 5-Fluoro-3′-[2-(ethylthio)ethyl]-3a′,6a′-dihydro-2′H-spiro[indole-3, 1′-pyrrol[3,4-c]pyrrol]-2,4;6′(1H,3′H,5′H)-trione (SI-183F)

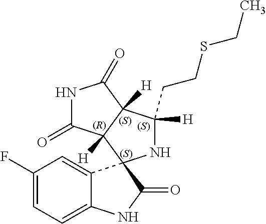

This compound prepared, using techniques, similar to those, described for synthesis of the Example 1, stages 1 and 2, and instead of 1-p-tolyl-pyrrol-2,5-dione L-methionine and isatin, pyrrol-2,5-dione, L-ethionine and 5-fluoro-1H-indole-2,3-dione were used, respectively. Yield: 80%. White crystal powder with melting temperature 285-287° C. (with decomposition, n-BuOH). LC/MS m/e 363 (m+H)⁺. ¹H NMR (200 MHz, DMSO-d₆, TMS): 11.34 (1H, br. s., NH), 10.37 (1H, s, NH), 7.03 (1H, td, J=9 and 3 Hz, F—CH—), 6.67-6.83 (2H, m, Ar), 4.33 (1H, br. s., 3′-CH), 4.04-4.20 (1H, m, 2′-NH), 3.67-3.90 (2H, m, 3a′-CH), 3.37-3.49 (3H, m, 6a′-CH), 2.52-2.67 (3H, m, CH₂CH₂SCH₂CH₃), 2.31-2.43 (1H, m), 1.98 (1H, dd, J=14 and 7 Hz, CH₂CH₂SCH₂CH₃), 1.64 (1H, dt, J=14 and 7 Hz, CH₂CH₂SCH₂CH₃), 1.26 (3H, c, CH₂CH₂SCH₂CH₃). Calculated, %: C, 56.19; H. 4.99; N. 11.56; S. 8.82. C₁₇H₁₈FN₃O₃S. Found. %: C. 63.55; H. 5.34; N. 9.26; S. 7.06.

### (04) Example 15

### 5-Bromo-3′-[2-(methylthio)ethyl]-3a′,6a′-dihydro-2′H-spiro[indolo-3,1′-pyrrolo[3,4-c]pyrrol]-2,4′,6′(1H,3′H, 5′H)-trione

### S-Bromo-3′-[2-methylthio)ethyl]-3a′,6a′-dihydro-2′H-spiro[indole-3,1′-pyrrolo[3,4-c]pyrrol]-2,4′,6′(1H,3′H, 5′H)-trione (SI-173N)

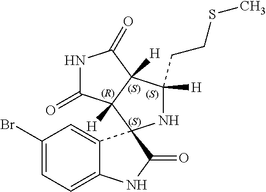

This compound prepared, using techniques, similar to those, described for synthesis of the Example 1, stages 1 and 2, and instead of 1-p-tolyl-pyrrol-2,5-dione and isatin pyrrol-2,5-dione and 5-bromo-isatin were used, respectively. Yield: 75%. White crystal powder with melting temperature 225-226° C. (n-BuOH). LC/MS m/e 410 (m+H)⁺. ¹H NMR (400 MHz, DMSO-d₆, TMS) □: 11.40 (br. s., 1H), 10.50 (br. s., 1H), 7.40 (d, J=8.3 Hz, 1H), 7.08 (s, 1H), 6.77 (d, J=7.9 Hz, 1H), 4.15 (br. s., 1H), 3.87 (br. s., 1H), 3.44 (t, J=7.7 Hz, 1H), 3.21-3.32 (m, 2H), 2.50-2.66 (m, 3H), 2.01-2.14 (m, 4H), 1.58-1.75 (m, 1H), 1.05 (d, J=6.2 Hz, 1H). Calculated, %: C, 46.84; H. 3.93; N. 10.24; S. 7.82. C₁₆H₁₆BrN₃O₃S. Found. %: C. 46.85; H. 3.95; N. 10.25; S. 7.83.

### (05) Example 16

### 3′-[2-(Methylthio)ethyl]-3a′,6a′-dihydro-2′H-spiro[indolo-3,1′-pyrrolo[3,4-c]pyrrol]-2,4′,6′(1H,3′H 5′H)-trione

### 3′-[2-(Methylthio)ethyl]-3a′,6a′-dihydro-2′H-spiro[indole-3,1′-pyrrolo[3,4-c]pyrrol]-2,4′,6′(1H,3′H,5′H)-trione (SI-148N)

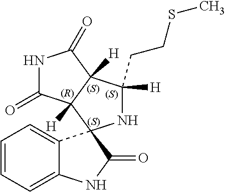

This compound prepared, using techniques, similar to those, described for synthesis of the Example 1, stages 1 and 2, and instead of 1-p-tolyl-pyrrol-2,5-dione pyrrol-2,5-dione was used. Yield: 89%. White crystal powder with melting temperature 234-236° C. (i-PrOH). LC/MS m/e 331 (m+H)⁺. ¹H NMR (500 MHz, DMSO-d₆, TMS) □: 11.32 (br. s., 1H), 10.33 (s, 1H), 7.20 (t, J=7.5 Hz, 1H), 6.96 (d, J=7.3 Hz, 1H), 6.90 (t, J=7.5 Hz, 1H), 6.79 (d, J=7.8 Hz, 1H), 4.35 (br. s., 1H), 4.14-4.22 (m, 1H), 3.70-3.83 (m, 2H), 3.44 (t, J=7.5 Hz, 1H), 3.23 (d, J=7.8 Hz, 1H), 2.55-2.70 (m, 3H), 1.98-2.11 (m, 4H), 1.68 (dd, J=14.0, 5.7 Hz, 1H), 1.05 (d, J=6.2 Hz, 6H) ¹³C NMR (DMSO-d₆) □: 180.9, 178.5, 176.8, 142.6, 129.4, 128.3, 126.5, 121.5, 109.6, 68.0, 62.5, 56.9, 53.6, 49.8, 40.6, 40.5, 40.4, 40.3, 40.3, 40.2, 40.1, 40.0, 39.9, 39.8, 39.7, 39.5, 31.6, 31.5, 25.9, 15.2. Calculated, %: C, 57.99; H. 5.17; N. 12.68; S. 9.68. C₁₆H₁₇N₃O₃S. Found. %: C. 58.00; H. 5.17; N. 12.69; S. 9.69.

### (06) Example 17

### 1-Methyl-3′-[2-(ethylthio)ethyl]-3a′,6a′-dihydro-2′H-spiro[indolo-3,1′-pyrrolo[3,4-c]pyrrol]-2,4′,6′(1H,3′H,5′H)-trione

### 1-Methyl-3′-[2-(ethylthio)ethyl]-3a′,6a′-dihydro-2′H-spiro[indole-3,1′-pyrrolo[3,4-c]pyrrol}-2,4′,6′(1H,3′H,5′H)-trione (SI-179)

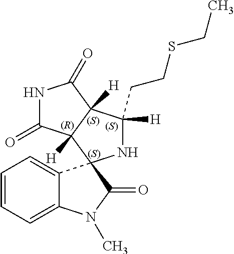

This compound prepared, using techniques, similar to those, described for synthesis of the Example 1, stages 1 and 2, and instead of 1-p-tolyl-pyrrol-2,5-dione and isatin, pyrrol-2,5-dione and 1-methyl-isatin were used, respectively. Yield: 75%. White crystal powder with melting temperature 217-218° C. (n-BuOH). LC/MS m/e 359 (m+H)⁺. ¹H NMR (500 MHz, DMSO-d₆, TMS) □: 11.34 (br. s., 1H), 7.27-7.36 (m, 1H), 6.91-7.10 (m, 3H), 4.20 (d, J=6.7 Hz, 1H), 3.77 (br. s., 1H), 3.47 (t, J=7.5 Hz, 1H), 3.30-3.39 (m, 1H), 3.23 (d, J=7.8 Hz, 1H), 3.10 (s, 3H), 2.57-2.69 (m, 2H), 1.97-2.08 (m, 1H), 1.61-1.70 (m, 1H), 1.14-1.26 (m, 3H). ¹³C NMR (DMSO-d₆) □: 178.8, 178.4, 176.7, 144.1, 129.6, 127.5, 126.2, 122.1, 108.6, 67.7, 57.1, 53.7, 49.8, 40.5, 40.4, 40.2, 40.0, 39.9, 39.7, 39.5, 32.1, 29.0, 26.2, 25.4, 15.3. Calculated, %: C, 60.15; H. 5.89; N. 11.69; S. 8.92. C₁₈H₂₁N₃O₃S. Found. %: C. 60.14; H. 5.90; N. 11.70; S. 8.93.

### (07) Example 18

### Preparation of L-Ascorbic Salt and 5′-(4-methylphenyl)-3′-[2-(methylthio)ethyl]-3a′,6a′-dihydro-2′H-spiro[indole-3,1′-pyrrolo[3,4-c]pyrrol]-2,4′,6′(1H,3′H,5′H)-trione

The load of 5′-(4-methylphenyl)-3′-[2-(methylthio)ethyl]-3a′,6a′-dihydro-2′H-spiro[indole-3,1′-pyrrolo[3,4-c]pyrrol]-2,4′,6′(1H,3′H,5′H)-trione (10 g, 24 mmol) in 540 ml of ethanol 96% was heated at agitation on water heater to clear solution, to which equimolar amount of L-ascorbic acid (4.18 g) was added, dissolved in 105 ml of treated water (calculated for 1 g in 25 ml of treated water). Prepared solution was carefully evaporated on rotor evaporator in vacuum resulting in light yellowish salt. Salt was recrystallized as needed from the mixture water:alcohol (1:4). Yield was quantitative. Melting T. 178° C. (with decomposition). LC/MS m/e 421 (M+H)⁺, 176 (M+H)⁺. Calculated, %: C, 58.28; H. 5.23; N. 7.03; S. 5.37. Found. %: C. 58.29; H. 5.24; N. 7.04; S. 5.38.

### (08) Example 19

### Preparation of Succinic Acid Salt and 5′-(4-methylphenyl)-3′-[2-(methylthio)ethyl]-3a′,6a′-dihydro-2′H-spiro[indole-3,1′-pyrrolo[3,4-c]pyrrol]-2,4′,6′(1H,3′H,5′H)-trione

The load of 5′-(4-methylphenyl)-3′-[2-(methylthio)ethyl]-3,a′,6a′- [symbol] -2′H-spiro[indole-3,1′-pyrrolo[3,4-c]pyrrol]-2,4′,6′(1H,3′H,5′H)-trione (10 g, 24 mmol) in 540 ml of ethanol 96% was heated at agitation on water heater to clear solution, to which equimolar amount of succinic acid (2.83 g) was added and agitated to dissolution. Prepared solution was carefully evaporated on rotor evaporator in vacuum resulting in off white salt with slightly creamy with huing. Salt was recrystallized as needed from the mixture water:alcohol (1:4). Melting T. 179-182° C. (with decomposition). LC/MS m/e 421 (m+H)⁺, 118 (m+H)⁺. Yield was quantitative. Calculated, %: C, 60.10; H, 5.42; N. 7.79; S. 5.94. Found. %: C. 60.12; H. 5.43; N. 7.78; S. 5.92.

### (09) Example 20

### Specific Activity to Human 11β-HSD1

The screening of spiro[pyrrolidineo-3,2-oxindole] of general Formula I or II by precision molecular docking on human 11β-HSD1 oxidoreductase 3D-model (PDB ID 3BZU available (Berman, et al. Announcing the worldwide Protein Data Bank, Nat. Struct. Biol. 10 (12) (2003) 980; R. A. Schweizer, A. G. Atanasov, B. M. Frey, A. Odermatt, A rapid screening assay for inhibitors of 11β-hydroxysteroid dehydrogenases (11β-HSD): flavanone selectively inhibits 11β-HSD1 reductase activity, Mol. Cell. Endocrinol. 212 (1-2) (2003) 41-49)) using software package AutoDock 4.2 and AutoDock Vina, revealed the ability of claimed compound of the Formula I to inhibit said enzyme which is a key one in extrarenal tissue-specific glucocorticosteroid metabolism and well-established target for designing of antidiabetic drugs (Schuster et al. J. Steroid Biochemistry & Molecular Biology 125 (2011)-P. 148-161).

1318 hypothetic structures were subjected to docking procedure using AutoDock software complex, among them for 1305 structures less free energy values were obtained for interaction with 11β-HSD1 A and B molecule site (E_{Doc}=−5.47 kkal/mol) then for the complex of such well-known non-steroid 11β-HSD1 inhibitor as S-28, and 13 compounds were found as non-active, that is had free energy values from −0.2 to −5.46 kkal/mol for A 11β-HSD1 site, and from −0.9 to −5.39 kkal/mol-for B 11β-HSD1 site. For base with 1318 structures 95.91% structures were active, that is had free energy values from −11.37 to −5.48 kkal/mol for A site, and from −10.93 to −5.51 kkal/mol-for B site. The most active were compounds containing residuals of aliphatic sulphur-containing amino acids with 2′ position of pyrrolidine cycle and had nanomolar inhibition constants. Thus, for example, SI-86 compound exhibit inhibiting activity to 11β-HSD1 with enzyme inhibition constant Kᵢ=4.14 nm (E, kkal/mol −8.87 and −8.54 at T=298.15 KJ, which exceeds similar index of known nonsteroid inhibitors (Table 1).

TABLE 1
Activity to human 11β-HSD1

| Compounds | Kᵢ, nm |
|---|---|
| SI-86 | 4.14 ± 0.25 |
| SI-34 | 4.41 ± 0.32 |
| SI-176N | 2.55 ± 0.22 |
| SI-0076 | 9.76 ± 1.11 |
| AMG 221 | 12.80 ± 1.70 |

### Example 21

### Evaluation of Anti-Ischemic Mnemotropic Properties of Exemplary Compounds SI-86 at Model Apoplectic Shock

In studies of rats with intracerebral bleeding of moderate severity modeled by autoblood injections (20 μl/100 g) into internal brain capsule it was established that administration of 3,2′-spiro-pyrrolo-2-oxindole derivative (SI-86 compound) at a dose of 10 mg/kg intraventricularly in therapeutic regimen (1 hour after stroke recovery and then every 24 h for 21 days) was more effective then intraperitoneal administration of citicoline (250 mg/kg), actovegin (16 mg/kg) and pyracetam (400 mg/kg), reduced case mortality and neurological deficit in acute and recovery stroke periods, and also improved mnestic functions. SI-86 compound was compared to mexidol (100 mg/kg intraperitoneally) by values of their cerebroprotective properties. Data obtained experimentally supported the utility of this substance as cerebroprotective agent.

Neuroprotective effect of 3,2′-spiro-pyrrolo-2-oxindole derivative (SI-86 compound) was studied on IUD model of moderate severity modeled under propofol anesthesia (60 mg/kg intraperitoneally (ip)) by autoblood administration into the brain internal capsule (stereotactic coordinates of the projection: H=7.0 mm, L=3.0 mm, A=1.5 mm from bregma) (20 ml/100 g) (Method for reproduction of intracerebral hemorrhage in rats/O. K. Yarosh, S. V. Kyrychenko, S. P. Halimonchyk [et al.]//Circulation and hemostasis.-2005.-No. 1.-P. 77-81). The chosen model allows to reproduce clinical pattern of ischemic stroke and is adequate for clinical study of potential neuroprotective agents.

As comparators the following drugs were used: mexydol (“Mexydol” UC Pharmasoft, Russia), 100 mg/kg; citicoline (“Somazina” Ferrer International, SA, Spain), 250 mg/kg; aktovegin (“Aktovegin”, Nycomed, Austria), 16 mg/kg pyracetam (“Pyracetam” Darnitsa, Ukraine), 400 mg/kg. According to the latest clinical guidelines regarding the treatment of patients with CVA approved by the Ministry of Health of Ukraine (Order No. 602 dated 3 Aug. 2012), all these drugs are allowed to be included into schemes for intensive patients therapy with CVA as neuroprotective agents. They were used at recommended doses for preclinical studies (McGrow C. P. Experimental Cerebral Infarction Effects of Pentobarbital in Mongolian Gerbils/C. P. McGrow//Arch. Neurol.-1977.-Vol. 34, No. 6.-P. 334-336). Experimental therapy of acute cerebral ischemia with SI-86 compound and comparator started 1 hour after IUD once a day for 21 days. The SI-86 derivative was investigated in conventionally effective dose of 10 mg/kg intraventricularly (iv)-dose that according to the results of our previous study provides maximum antihypoxic activity of SI-86 compound. Reference drugs were injected intraperitoneally (ip). Rats of control pathology group were injected by autoblood and as therapy 0.9% NaCI solution was administered calculated for 2 ml/kg iv.

Pseudo-operated rats were exposed to all interventions (anesthesia, craniotomy) excluding autoblood administration that leveled traumatic impact of experimental conditions.

Neurological deficit in animals with acute CVA respectively in acute (4th day) and recovery (21 st day) periods was measured on C. P. McGrow stroke-index scale [13]. The state severity was determined by amount of relevant points: to 3 points-mild, 3 to 7 points-average, above 7 points-severe degree. Pareses, paralyses of limbs, tremors, circus movement, ptoses, lateral positions, ability of rats to be maintained at the core of 15 cm diameter, rotating at the rate of 3 rpm were noted. The animals were tested daily by displaying the amount of points:

one-sided semi-ptosis—0.5 points;
one-sided ptosis—1 point;
tremor—0.5 points;
circus movements—0.5 points;
pareses of limbs (for each limb)—1 point;
paralyses of limbs (for each limb)—2 points;
lateral position—3 points;
disability to maintain on rotating rod during 4 minutes—3 points.

Evaluation of the ability of animals to learn and memorize of aversive stimulus was examined in the test of conditioned response of passive avoidance (CRPA) [2]. The technique is based on innate rat instinct to limited shadowy space. The study in rats was conducted in two-compartment unit consisted of two sections—light and dark. An animal was arranged into light compartment, fixing latency time for entering into the dark compartment, wherein rat received current irritation and ran in the light compartment. CRPA retention was tested every other day under latency time change of rat entering into the dark compartment. Also the number of animals was noted not fully entered the dark chamber. Cerebroprotective efficiency criteria of studied compounds were also animal death term (days) and their mortality rate (in %).

Any traumatic manipulations and euthanasia of animals by decapitation were performed under conditions of propofol anesthesia (“Fresenius Kabi”, Austria).

Quantitative data were processed using statistical StatPlus 2009 processing. The statistical significance of differences was assessed by Fisher's angular transformation (lethality outcome), also parametric t Student criterion were used in case of normal distribution of variation series, nonparametric W White criterion—in its absence.

As can be seen from the data presented in Table 2, at model BMC all of substances under study except pyracetam were useful in decreasing the mortality rate of animals in terms of pathological state, indicating that they have cerebroprotective effect. However, by protective effect value on ischemic brain they had certain qualitative differences. The most effective neuroprotective properties 3,2′-spiro-pyrrolo-2-oxindole derivative (SI-86 compound) (10 mg/kg iv), mexydol (100 mg/kg iv) and citicoline (250 mg/kg iv) demonstrated providing 100% cerebroprotective protection (in terms of 24 h observations of mortality rates in groups of animals treated with these drugs was 0% vs. 17.4% in controls). During 48 hours observation in rats under therapy with compounds SI-86 any deaths was not observed, in contrast to mexydol and citicoline treatment where mortality of WMC animals reached 7.9 and 8.5%, respectively. By the value of cerebroprotective action in specified period of experimental compound GI SI-86 (10 mg/kg iv) significantly exceeded mexydol (100 mg/kg iv), citicoline (250 mg/kg iv) and aktovegin (16 mg/kg iv). The course administration of 3,2′-spiro-pyrrolo-2-oxindole derivative, as well as mexydol, citicoline and actovegin within 4 days of therapy from the point of pathology reproduction provided the reduction of mortality in rats at the end of the observation period relative to control 18 6; 17.2; 15.1 and 9.3% in the average respectively (p<0.05). It should be noted that pyracetam had no significant impact on reducing mortality in animals with GI. Such a low efficiency of pyracetam under these conditions is consistent with ambiguous clinical results concerning its early prescription in CVA.

Thus, describing the research results of assessment for cerebroprotective action of 3,2′-spiro-pyrrolo-2-oxindole derivative (SI-86 compound), mexydol, citicoline and actovegin in terms of model of intracerebral hemorrhage it may be concluded that, to some extent, all of them have inherent protective effect on ischemic brain. The most neuroprotective activity was found in SI-86 compound (10 mg/kg iv) after 48 hours of observation, when by its efficiency it was statistically better than all reference drugs. By the value of cerebroprotective effect in said period studied GI drugs can be arranged in the following order: SI-86 compounds (10 mg/kg iv)>mexydol (100 mg/kg ip)>citicoline (250 mg/kg ip)>aktovegin (16 mg/kg ip)>pyracetam (400 mg/kg ip).

According to the data of literature, integrative indicators to assess the quality of the protective effect of potential neuroprotective agent in ischemic brain, along with decreasing in mortality, are rapid elimination of neurological deficit and improving memory functions. Therefore it was feasible to evaluate cerebroprotective properties of 3,2′-spiro-pyrrolo-2-oxindole derivative by the dynamics of neurological status in rat GI model

TABLE 2
Effect of 3,2′-spiro-pyrrolo-2-oxindole derivative (SI-86 compound) and
some cerebroprotective agents at intraperitoneal treating administration
on lethality of rats with intracerebral hemorrhage of average severity

| | Lethality, abs./% | | | | | | |
|---|---|---|---|---|---|---|---|
| | | Control | | | | | |
| | | pathology | | | | | |
| | Pseudooperated | CH + 2 ml/kg | | CH + | CH + | CH + | CH + |
| | animals + 2 ml/kg | 0.9% | CH + SI-86 | mexydol | citocoline | actovegin | pyracetam |
| | 0.9% | NaCI, | (10 mg/kg, | (100 mg/kg), | (250 mg/kg), | (16 mg/kg), | (400 mg/kg), |
| Period, h | NaCI, ipn = 30 | ipn = 46 | iv), n = 34 | n = 38 | n = 47 | n = 47 | n = 41 |
| 12 | 0/0% | 4/8.7%° | 0/0% | 0/0%*^{#} | 0/0%*^{#} | 0/0%*^{#} | 2/4.9%° |
| 24 | 0/0% | 8/17.4%° | 0/0%*^{#} | 0/0%*^{#} | 0/0%*^{#} | 1/6.4%*^{#} | 6/14.6%° |
| 48 | 0/0% | 10/21.7%° | 0/0%*^{#&•$} | 3/7.9%°*^{#} | 4/8.5%°*^{#} | 4/8.5%°*^{#} | 9/22%° |
| 72 | 0/0% | 12/26.1%° | 2/5.9%°*^{#} | 4/10.5%°*^{#} | 6/12.8%°* | 7/21.1%°* | 10/24.4%° |
| 96 | 0/0% | 14/30.4%° | 4/11.8%°*^{#} | 5/13.2%°* | 7/14.9%°* | 8/21.1%° | 11/27.5%° |

Notes:
1. IG - intracerebral hemorrhage;
2. °p < 0.05 in relation to pseudo-operated animals;
3. *p < 0.05 in relation to control;
4. ^{#}p < 0.05 in relation pyracetam (400 mg/kg ip);
5. ^{&}p < 0.05 in relation to mexydol (100 mg/kg ip);
6. ^{•}p < 0.05 in relation to citocoline (250 mg/kg ip);
7. ^{$}p < 0.05 in relation to actovegin (16 mg/kg ip)

TABLE 3
Effect of 3,2′-spiro-pyrrolo-2-oxindole derivative (SI-86 compound) on
neurological deficit in rats with intracerebral hemorrhage (m ± m, n = 15)

| | Period, day | |
|---|---|---|
| Animal groups | 4 | 21 |
| Pseudo-operated animals + 2 ml/kg | 0.0 ± 0.0 | 0.0 ± 0.0 |
| 0.9% NaCI, ip | | |
| Control pathology CH + 2 ml/kg | 7.40 ± 0.33° | 4.70 ± 0.25° |
| 0.9% NaCI, ip | | |
| CH + SI-86 (10 mg/kg, iv) | 4.10 ± 0.32°*^{#•$} | 3.10 ± 0.33°*^{#&$} |
| CH + mexydol (100 mg/kg, ip) | 4.30 ± 0.25°*^{#•$} | 3.80 ± 0.21°*^{#•$} |
| CH + citocoline (250 mg/kg, ip) | 5.00 ± 0.18°*^{#$} | 3.10 ± 0.15°*^{#&$} |
| CH + actovegin (16 mg/kg, ip) | 5.80 ± 0.15°*^{#} | 4.20 ± 0.25°*^{#} |
| CH + pyracetam (400 mg/kg, ip) | 6.90 ± 0.39° | 4.10 ± 0.15°* |

Notes:
1. CH—cerebral hemorrhage; iv—intraventricularly; ip—intraperitoneally;
2. °p < 0.05 in relation to psuedo-operated animals;
3. *p < 0.05 in relation to control pathology;
4. ^{#}p < 0.05 in relation to pyracetam (400 mg/kg ip);
5. ^{•}p < 0.05 in realtion to citocoline (250 mg/kg ip);
6. 7. ^{$}p < 0.05 in relatin to actovegin (16 mg/kg ip)

Experimental treatment of CVA rats with SI-86 compound, as well as with mexydol, citocoline and actovegin, was benefit for neurological status improvement starting from the very first days of cerebral ischemia (Table 3). Having analyzed the dynamics of neurological deficit regress it could be noted that in early CVA period 3,2′-spiro-pyrrolo-2-oxindole derivative were statistically better than citocoline and actovegin, comparing it with mexydol: on the 4^{th} day of observation the average point under C. P. McGrow scale was 4.1 in comparison with 5.0; 5.8 and 6.9 (p<0.05). During recovery GI model period the leaders were SI-86 compound and citocoline. They demonstrated the similar ability to reduce neurological symptoms—average point under C. P. McGrow scale was 3.1 in comparison with 4.7 in control group and 3.8; 4.2 and 4.1 on the background of mexydol, actovegin and pyracetam administration (Table 4). Regarding recovery of mnestic functions in late GI period, SI-86 compound was statistically better than all studied reference preparations for improvement of studied indices of URPU test (Table 4).

TABLE 4
Effect of 3,2′-spiro-pyrrolo-2-oxindole derivative (compounds SI-86) at
treating administration on learning and memory in rats with
intracerebral hemorrhage on the 21^{st} day of the experiment by the test of
conditional reaction of passive avoiding (m ± m, n = 15)

| | Latent period for entrance into dark | |
|---|---|---|
| | compartment, s | |
| Animal group | Prior study | 24 hours after study |
| Pseudo-operated animals + 2 ml/kg | 5.20 ± 0.35 | 219.1 ± 2.53 |
| 0.9% NaCI, ip | | |
| Control pathology CH + 2 ml/kg | 19.90 ± 0.57 | 42.50 ± 0.88° |
| 0.9% NaCI, ip | | |
| CH + SI-86 (10 mg/kg, iv) | 9.50 ± 0.34°*^{#•$} | 127.10 ± 2.71°*^{#•$} |
| CH + mexydol (100 mg/kg, ip) | 11.30 ± 0.47°*^{#$} | 106.80 ± 2.77°*^{#•$} |
| CH + citocoline (250 mg/kg, ip) | 12.50 ± 0.75°*^{#} | 106.3013.87*^{#$} |
| CH + actovegin (16 mg/kg, ip) | 14.90 ± 0.48°*^{#} | 87.70 ± 3.07*^{#} |
| CH + pyracetam (400 mg/kg, ip) | 15.90 ± 0.51°* | 71.10 ± 2.21* |

Notes:
1. CH—cerebral hemorrhage; iv—intraventricularly; ip—intraperitoneally;
2. °p < 0.05 in relation to pseudo-operated animals;
3. *p < 0.05 in relation to control pathology;
4. ^{#}p < 0.05 in relation to pyracetam (400 mg/kg ip);
5. ^{•}p < 0.05 in relation to citocoline (250 mg/kg ip);
6. ^{$}p < 0.05 in relation to actovegin (16 mg/kg ip)

By the ability to improve mnestical function in GI model recovery period all substances can be arranged in the following order: SI-86 (10 ms/kg ip)≧mexydol (100 mg/kg ip)≧citocoline (250 mg/kg ip)≧actovegin (16 mg/kg ip) pyracetam (400 mg/kg ip).

Original SI-86 derivative significantly reduces mortality and neurological deficit in rats with acute and recovery period of intracerebral hemorrh

### Example 22

### Evaluation of Corrective Effect of 3,2′-spiro-pyrrolo-2-oxindole Derivative (Compounds SI-86) on the Dynamics of Corticosterone and Steroid Excitotoxicity

The neuroprotective effect of 3,2′-spiro-pyrrolo-2-oxindole derivative (SI-86 compound) was studied in Wistar rats on a model of cerebrovascular accident (CVA) of ischemic type in archencephalic pool forming bilateral carotid occlusion (BCO) by carotid artery ligation. The ligations for common carotid arteries was imposed under propofol anesthesia at a dose of 60 mg/kg intraperitoneally (ip), using surgical access to the front of the neck cutting through its white line. The chosen model allows to reproduce the clinical pattern of ischemic stroke and is adequate for clinical study of potential neuroprotective substances (Drug discovery and Evaluation: pharmacological assays/H. Gerhard Vogel (ed.).-2nd ed. 1453p.).

As a comparator citocoline was used (“Somazina” Ferrer Snternathional, SA, Spain) in recommended dose for preclinical studies of 250 mg/kg ip (Preclinical Study of Specific Activity of Potential Neuroprotective Drugs: Method. Recommendations/[Y. S. Chekman, Yu. Y. Hubskyy, I. F. Belenychev et al.].-Kiev, 2010.-81 s). Experimental therapy of acute cerebral ischemia with SI-86 compound and citocoline was started 1 hour after BCO, and then once a day for 21 days. The derivative of 3,2′-spiro-pyrrolo-2-oxindole was investigated in conventionally effective dose of 10 mg/kg intraventricularly (iv)—dose which is provided maximum antihypoxic activity of SI-86 compound by the results of our previous study. The reference drugs were injected intraperitoneally (ip). BCO was applied to the rats of control pathology and 0.9% NaCI solution was administered as therapy calculated for 2 ml/kg ip.

Pseudo-operated rats were exposed to all interventions (anesthesia, skin incision, dissection of vessels) except arteries ligation that leveled traumatic impact of experimental conditions.

To determine a corticosterone level (species-specific cortisol analogue in rats, which is also a substrate for 11β-HSD1) in the corresponding period (4 th and 21 st CVA day) in rat by sagittal sinus puncture, blood was sampled (0.2-0.4 ml). The cortisol level was measured by ELISA using a CORTICOSTERONEE KIT set (Germany) on the instrument of the company “Hipson” (Czech Republic).

To establish the effect of course administration of SI-86 compound on steroid synthesis in animals without cerebral ischemia the dynamics of its level in pseudo-operated rats in the same period was additionally determined.

An operative intervention (skin incision, dissection of vessels) was accompanied by increasing corticosterone levels in relation to intact animals (no traumatic manipulation were made for them) as evidenced by the preservation of its high titer (average 33.6%) even on the 4th day after operation (p<0.05). Such an increasing of 11β-corticosterone can be explained by stress reaction of animals to injury and is typical for the early postoperative period. Subsequently (21 st day of the experiment) the normalization of the studied hormone was observed and its blood content did not differ from intact rats (Table 5)

TABLE 5
Effect of SI-86 compound on dynamics of 11β-corticosterone (ng/ml) level
in venous blood in rats having bilateral carotid occlusion (m ± m, n = 5-7)

| | Period, day | |
|---|---|---|
| Animal group | 4 | 21 |
| Intact animals | 90.04 ± 4.32 | |
| Pseudo-operated animals + 0.9% | 120.25 ± 2.05°* | 93.20 ± 3.59*^{•} |
| NaCI (2 ml/Kg) | | |
| Pseudo-operated animals + SI-86 | 81.95 ± 0.61* | 83.02 ± 4.25* |
| (10 mg/kg iv) | (−31.9%) | (−10.2%) |
| Control pathology BCO + 0.9% | 468.00 ± 14.07° | 337.19 ± 7.90°^{•} |
| NaCI (2 ml/kg ip) | (+289.2%) | (+261.8%) |
| BCO + SI-86 (10 mg/kg iv) | 186.12 ± 1.43°*^{#} | 144.67 ± 3.43°*^{#•} |
| | (+54.8%) | (+55.2%) |
| | [−60.2%] | [−57.1%] |
| BCO + citocoline (250 mg/kg ip) | 272.79 ± 1.95°* | 235.52 ± 7.82°*^{•} |
| | (+126.9%) | (+152.7%) |
| | [−41.7%] | [−30.2%] |

Notes:
1. BCO—bilateral carotid occlusion; iv—intraventricularly; ip—intraperitoneally;
2. °p < 0.05 in relation to intact animals;
3. *p < 0.05 in relation to control animals during corresponding observation period;
4. ^{#}p < 0.05 in relation to citocoline;
5. ^{•}p < 0.05 in relation to 4 days in corresponding experimental group;
6. in round parenthesis—dynamics in percents in relation to the group of pseudo-operated animals corresponding observation period;
7. in square bracket—dynamics in percents in relation to control pathology group in corresponding observation period

The administration to pseudooperated rats of SI-86 compound (10 mg/kg iv) leveled the effect of traumatic manipulation indicating to stable blood concentrations of 1β-corticosterone comparable with this index in intact animals. This may indicate to the presence of stresprotective properties in investigated 3,2′-spiro-pyrrolo-2-oxindole derivative. The analysis of the corticosterone level in rats under BCO conditions showed that 96 hours (4th day) after modeling pathology, its level significantly increased in relation to the same index in pseudo-operated animals—by 3.9 times, while at the end of observation (21 st day), its titer remained elevated in 3.6 time (Table). Given that corticosterone was investigated in sagittal sinus blood in the brain, one can talk about certain probability of steroid excitotoxicity formation maintained during CVA regenerative period.

Obtained data are consistent with the results of other researchers who have studied the dynamics of corticosterone in different periods of acute cerebral ischemia and the development of neurodestructive diseases. Increasing of glucocorticoids level has morphogenetic effects on the functioning of neurons in the brain [Herbert J. et al., 2006; Goodyer I. M. et al., 2006]. In particular, high corticosterone levels in terms of CVA correlates with the reduction of neurons density in the hippocampus, neuroapoptosis initiation, development of significant neurological deficit, mnestical functions disorder and significant case mortality.

Therapeutic courses of administration to CVA animals of 3,2′-spiro-pyrrolo-2-oxindole derivative SI-86 compound (10 mg/kg iv), similar to citocoline (250 mg/kg ip), was accompanied by less intensive growth of corticosterone level. So, after 96 hours, its concentration in the sagittal sinus blood was decreased in relation to the control pathology group to 60.2% and 41.7%, respectively, and after 21 days—57.1% and 30.2%, respectively (p<0.05). Such action of these substances may indicate the presence of modulating effect on steroid excitotoxicity development. And, in acute cerebral ischemia, by the ability to reduce test hormone content both in CVA acute and in the recovery period, therapy with compounds SI-86's is statistically better than the citocoline administration, in 1.46 and 1.62 times, respectively. In our opinion, the presence of the corrective effect of SI-86 compound on glucocorticoid balance, may indicate its ability to prevent the development of destructive changes in ischemic brain, contribute to preservation of neuron structural integrity and consequently to reduce ischemia nidus and penumbra area. It should be noted that such action of 3,2′-spiro-pyrrolo-2-oxindole derivative is equally evident both in acute and in the recovery period of ischemia. Antisteroid effect of SI-86 compound in terms of CVA may be its main mechanism for cerebroprotective action. It is also important that the administration of studied substance only reduces elevated corticosterone level and its titer does not differ from physiological one even with course therapy. The latter points to the safety of its use.

Thus, the results indicate the major cellular pathogenetic ischemia units in terms of CVA (modulated impact on steroid excitotoxicity) perspective from the point of view of depth study of neuroprotective action of 3,2′-spiro-pyrrolo-2-oxindole derivative (SI-86 compound). These characteristics of SI-86 compounds in acute cerebral ischemia are desirable taking into account the possibility of its enteral administration and the presence of pathogenic effect on the primary ischemic cascade levels, there are all reasons for its possible prescription both to patients in different CVA periods and to patients with chronic cerebrovascular pathology.

### Example 23

### Positive Therapeutic Effect on the Dynamics of Neurodestruction Processes in Acute Cerebral Ischemia (the Dynamics of Neuronal-Specific Enolase (NSE) Activity)

For more grounded clarification of degree of its protective effect on brain in cerebrovascular accident (CVA), the effect of course therapy with 3,2′-spiro-pyrrolo-2-oxindole derivative (SI-86 compound) on intensity of destructive changes course was estimated in the neuron membranes by the dynamics of neuronal activity-specific enolase (NSE), which is an early marker of nervous tissue damage, NSE—is found mainly in neurons and neuroendocrine cells. In neurological diseases, including CVA, the outage of neuron-specific enzymes and their isoenzymes from damaged neurons is noted, allowing to study the depth and intensity of structural and functional abnormalities in the central nervous system of biomembranes in the early stages (Davalos A. Citicoline in the treatment of acute ischemic stroke: an international, randomised, multicentre, placebo-controlled study (ICTUS trial)/A. Davalos, J. Alvarez-Sabin, J. Castillo//Lancet.-2012.-380.-P. 349-357).

Studies in rats with model cerebral accident (bilateral carotid occlusion) found that administration of 3,2′-spiro-pyrrolo-2-oxindole derivative (SI-86 compound) at a dose of 10 mg/kg intraventricularly in therapeutic regimen (1 hours after stroke recovery and then every 24 hours within 21 days) was more effective than intraperitoneal administration of citocoline (250 mg/kg), reduces the activity of neuron-specific enolase, indicating the reduction by tested substance of neurodestructive changes in the brains of animals.

The experimental therapy of acute cerebral ischemia with compounds SI-86 and citocoline (“Somazina” Ferrer Snternathional, SA, Spain) started 1 hour after BCO, and then once a day for 21 days. The derivative of 3,2′-spiro-pyrrolo-2-oxindole was studied in conventionally effective dose of 10 mg/kg intraventricularly (iv)—dose that by the results of our previous study provided maximum antihypoxic activity of the SI-86compound. Reference drugs were injected intraperitoneally (ip). In our studies citocoline under experimental CVA conditions was injected ip in a dose recommended for preclinical studies of 250 mg/kg. BCO was made to rats of control pathology and as therapy 0.9% NaCI solution was administered calculated for 2 ml/kg ip.

Pseudooperative rats were exposed to all interventions (anesthesia, skin incision, dissection of vessels) except arteries ligation that leveled traumatic effect of experimental conditions.

To determine neurospecific enolase, specific marker of cerebral ischemia,—to the period (4 th and 21 st day of CVA) in rat's blood was sampled by sagittal sinus puncture (0.2-0.4 ml). NSE activity was measured by ELISA using a set NSE EIA KIT (DAI, USA) on the instrument of the company “Hipson” (Czech Republic).

Any traumatic manipulations and euthanasia of animals were performed by decapitation under conditions of propofol anesthesia.

Quantitative data were processed using statistical StatPlus 2009 processing. We used parametric t Student criterion in case of normal distribution of variation series, nonparametric criterion W White—in its absence.

Results and discussion. The analysis of the activity of studied markers in rats under BCO conditions showed that 96 hours (4th day) after pathology modeling, its level significantly increased in relation to the same index in pseudo-operated animals by 10.5 times, while at the end of observation (21 st day), NSE activity continued to remain high in almost 7.1 times (Table 6).

TABLE 6
Positive effect of course administration to rats with acute cerebral ischemia
of SI-86 compound on dynamics of neurodestructive changes (m ± m, n = 7)

| | Period, day | |
|---|---|---|
| Animal groups | 4 | 21 |
| Pseudo-operated animals + 2 ml/kg | 0.118 ± 0.001 | 0.110 ± 0.005 |
| 0.9% NaCI, ip | | |
| Control pathology BCO + 2 ml/kg | 1.238 ± 0.059* | 0.784 ± 0.033* |
| 0.9% NaCI, ip | | |
| BCO + SI-86 (10 mg/kg, iv) | 0.61 ± 0.004*^{#•} | 0.204 ± 0.015*^{#•} |
| BCO + citocoline (250 mg/kg, ip) | 0.972 ± 0.010*^{#} | 0.385 ± 0.010*^{#} |

Notes:
1. BCO—bilateral carotid occlusion; iv—intraventricularly; ip—intraperitoneally;
2. °p < 0.05 in relation to index of pseudooperated rats;
3. *p < 0.05 in relation to index of control pathology;
5. ^{•}p < 0.05 in relation to index of citocoline group

Our results regarding fluctuations of enolase activity in different stroke periods coincide with literature data [Rohlwink U K, Figaji A A. Biomarkers of Brain Injury in Cerebral Infections //Clin Chem. 2013 Oct. 29.]. Thus, according to the researchers, the significant increase in NSE in the acute phase of cerebral ischemia is mainly caused by neurondestruction due to the direct effects of ischemic factor on intracellular metabolism. In the later CVA period when adaptive and reparative processes are activated enolase activity is gradually decreased, but not reduced to normal figures.

This negative NSE activity dynamics in terms of CVA demonstrates not only significant value of ischemic focus, but also allows to predict with certain probability poor prognosis for a patient (lethal outcome, significant deterioration of cognitive and memory functions, loss of adaptive capacity to the environment, etc.).

The therapeutic course administration to CVA animals of 3,2′-spiro-pyrrolo-2-oxindole derivative compounds SI-86 (10 mg/kg iv), similar to citocoline (250 mg/kg ip), was accompanied by less intensive NSE activity increasing. So, in 96 hours, enzyme activity decreased in relation to the control pathology group by 2.03 and 1.27 times respectively, and in 21 days—by 3.84 and 2.03 respectively (p<0.05). This action of test drugs evidences about the existence of their cytoprotective effect. And, in acute cerebral ischemia, by their ability to reduce neurodestructor marker activity both in CVA acute and in the recovery period, the effectiveness of therapy with SI-86 compound is statistically better than in case of citocoline administration by 1.6 and 1.9 times respectively. Positive NSE activity dynamics against the background of course administration of SI-86 compound indicates to its ability to prevail development of destructive changes in ischemic brain, promote preservation of structural integrity of neurons and consequently reduce ischemia focus and penumbra area. It is also important that the cytoprotective effects of 3,2′-spiro-pyrrolo-2-oxindole derivative were revealed similar both in acute and in the recovery period of ischemia.

### Example 24

### Comparative Evaluation of Effect of 3,2′-spiro-pyrrolo-2-oxindole Derivatives (SI-86 Compound) on the Dynamics of Neurodestructive Changes in Intracerebral Hemorrhage Model (by Dynamics of NSE Activity)

Studies in rats with intracerebral hemorrhage of moderate severity modeled by autoblood injection into the internal brain capsule (20 ml/100 g) found that administration of 3,2′-spiro-pyrrolo-2-oxindole derivative (SI-86 compound) at a dose of 10 mg/kg intraventricularly in therapeutic regimen (1 hour after stroke recovery and then every 24 hours within 21 days) were more effective than citocoline intraperitoneal administration (250 mg/kg), reduces the activity of neuron-specific enolase indicating the weakening by tested substance of neurodestructive changes in the brains of animals.

The experimental therapy of acute cerebral ischemia with SI-86 compound and citocoline was started 1 hour after BCO, and then once a day for 21 days. The derivative of 3,2′-spiro-pyrrolo-2-oxindole was investigated in conventionally effective dose of 10 mg/kg intraventricularly (iv)—dose that according to the results of our previous studies provided maximum antihypoxic activity of SI-86 compound. Reference drugs were injected intraperitoneally (ip). In our studies citocoline under conditions of experimental CVA injected ip in a dose recommended for preclinical studies of 250 mg/kg [Chekman I. S. al., 2010; Khodakivskii O. A., Chereshniuk I. L., 2013]. Autoblood was injected to rats with control pathology and 0.9% NaCI solution was administered as therapy calculated for 2 ml/kg ip.

Psevdooperated rats were exposed to all interventions (anesthesia, craniotomy) with the exception of autoblood administration that leveled traumatic impact of experimental conditions.

To determine neuron-specific enolase, the specific marker of cerebral ischemia,—for corresponding period (4 th and 21 st day of CVA) in rats, blood was sampled by sagittal sinus puncture (0.2-0.4 ml). NSE activity was measured by ELISA using a set NSE EIA KIT (DAI, USA) on the instrument of the company “Hipson” (Czech Republic).

Any traumatic manipulations and euthanasia of animals by decapitation were performed under conditions of propofol anesthesia.

Quantitative data were processed using StatPlus 2009 statistical processing. We used parametric t Student criterion in case of normal distribution of variation series, nonparametric W White criterion—in its absence.

The analysis of the activity of studied marker in rats under IUD conditions showed that 96 hours (4th day) after modeling pathology, its level significantly increased in relation to the same index in pseudo-operated animals by 18.9 times, while at the end of observation (21 st day), NSE activity continued to remain high in almost 8.8 times (Table 7).

TABLE 7
Effect of course administration of SI-86 compound
to rats on dynamics of neurodestructive changes in
rats with acute cerebral hemorrhage (m ± m, n = 7)

| | Period, day | |
|---|---|---|
| Animal groups | 4 | 21 |
| Pseudo-operated animals + | 0.149 ± 0.008 | 0.140 ± 0.015 |
| 2 ml/kg 0.9% NaCI, ip | | |
| Control pathology BCO + | 2.816 ± 0.048* | 0.947 ± 0.017* |
| 2 ml/kg 0.9% NaCI, ip | | |
| BCO + SI-86 (10 mg/kg, iv) | 1.065 ± 0.019*^{#•} | 0.260 ± 0.014*^{#•} |
| BCO + citocoline (250 mg/kg, ip) | 1.292 ± 0.069*^{#} | 0.309 ± 0.05*^{#} |

Notes:
1. CH—cerebral hemorrhage; iv—intraventricularly; ip—intraperitoneally;
2 *p < 0.05 in relation to index of pseudooperated rats;
3. ^{#}p < 0.05 in relation to index of control pathology;
5. -p < 0.05 in relation to index of citocoline group

Our results regarding fluctuations of enolase activity in different periods of stroke coincide with literature data [A. K. Piskunov, 2010; Grishanov T. G. et al., 2011]. Thus, according to the researchers, the significant increase in NSE in the acute phase of cerebral ischemia is caused mainly by neuron destruction due to the direct effects of ischemic factor on intracellular metabolism. In the later CVA period when adaptive and reparative processes are activated, enolase activity is gradually decreased, but not reduced to normal figures.

This negative NSE activity dynamics under CVA conditions demonstrates not only significant amount of ischemic focus, but also allows the certain probability to predict poor prognosis for a patient (lethal outcome, significant deterioration of cognitive and memory functions, loss of adaptive capacity to the environment, etc.) [Kladovo E. A., et al., 2011; Grishanov T. G. et al., 2011].

The therapeutic course administration to CVA animals of 3,2′-spiro-pyrrolo-2-oxindole derivative SI-86 compound (10 mg/kg iv), similar to citocoline (250 mg/kg ip), was accompanied by less intensive NSE activity increasing. So, in 96 hours. enzyme activity was decreased in relation to the control pathology group by 2.6 and 2.2 times respectively, and in 21 days—by 3.6 and 3.1 times, respectively (p<0.05). Said action of test substances may indicate the presence of their cytoprotective effect. And, in acute cerebral ischemia, by the ability to reduce the neurodestruction marker activity both in CVA acute and in the recovery period, the efficiency of therapy with SI-86 compound is statistically better than citocoline administration, by 17.6% and 15.9%, respectively. In our view, the positive NSE activity dynamics against the background of course administration of SI-86 compounds indicates to its ability to prevent the development of destructive changes in ischemic brain, promote the preservation the structural integrity of neurons and consequently reduce ischemia focus and penumbra area. It is also important that the cytoprotective effects of 3,2′-spiro-pyrrolo-2-oxindole derivative were similar both in acute and in recovery period of ischemia. Thus, the derivative of 3,2′-spiro-pyrrolo-2-oxindole SI-86 compound in terms of the hemorrhagic stroke model reveals properties of both primary and secondary cerebroprotector.

### Example 25

### Assessment of Acute Toxicity

The acute toxicity study was conducted when administered compounds SI-86 to rats into the stomach at doses of 3500 and 4000 mg/kg. Each dose was tested in 4 animals. The duration of observation of rats after administration of test compound was 2 weeks. The results are in Table 8.

TABLE 8
Acute toxicity parameters for SI-86 at
single peritoneal administration in rats

| Doses tested, | Number of | Effect |
|---|---|---|
| mg/kg | animals | (died/total) |
| 3500 | 4 | 0/4 |
| 4000 | 4 | 1/4 |

A dose of 3500 mg/kg did not cause significant variations in the general state of rats, all animals survived. With increasing doses up to 4000 mg/kg one rat of 4 died (25%). The lethal outcome took place within 12 hours and accompanied by symptoms that showed the effect of SI-86 on the central nervous system (lateral position, respiratory failure).

According to the Hodge and Sterner classification, SI-86 compound can be attributed to low-toxic substances (IV class of toxicity) as its LD₅₀ when administered intraventricularly, is within the range of 501-5000 mg/kg.

### Example 26

### Evaluation of Antihypoxic Activity

The data obtained during the preliminary screening of original 3,2′-spiro-pyrrolo-2-oxindole derivatives allowed to identify compounds, which exhibit sufficiently high antihypoxia activity on CVA model (Table 9). Thus, the preventive administration of compounds under the designation SI-86 and SI-108 in the same dose of 10 mg/kg iv, as well as mexydol (100 mg/kg ip), significantly increased the life duration in rats in relation to control 33.7; 28.6 and 80.2% in average respectively. Other substances at a dose of 10 mg/kg had no significant effect on the increase of life duration of animals which may indicate to the lack of their antihypoxic activity under said pathological state.

TABLE 9
Effect of administration of compounds SI-108, SI-86 and
mexydol onduration of rat heart bioelectrical activity
under acute asphyxia conditions

| | | | Duration of | |
|---|---|---|---|---|
| | | Number | heart | |
| Test conditions, | | of | bioelectrical | Antihypoxic |
| preparations | Dose | animals | activity, min | activity, % |
| Control (0.9% | 2 ml/kg ip | 15 | 11.6 ± 0.7 | — |
| NaCI solution) | | | | |
| SI-108 | 10 mg/kg iv | 7 | 9.5 ± 1.1 | −18.4 |
| SI-86 | 5 mg/kg iv | 7 | 13.0 ± 1.5 | +12.1 |
| SI-86 | 10 mg/kg iv | 7 | 12.3 ± 1.4 | +5.8 |
| SI-86 | 15 mg/kg iv | 7 | 11.3 ± 1.0 | −2.8 |
| Mexydol | 100 mg/kg ip | 7 | 17.5 ± 0.5* | +50.9 |

The study of antihypoxic activity of 10 original derivatives of 3,2′-spiro-pyrrolo-2-oxindole was conducted on models of acute normobaric hypoxic hypoxia with hypercapnia (ANHHH) and acute asphyxia. ANHHH was modeled by means of rat arrangement into isolated pressure compartments (0.001 m³). The observation conducted until the death of the animals. The antihypoxic activity was assessed by life duration (in minutes) in relation to the control taken as 100%, by the formula AA=tt/tk×100%, wherein AA—antihypoxic activity (%), tt—life duration of test animals, tc—life duration of control animals.

Acute asphyxia was modeled in rats anesthetized with propofol (60 mg/kg) intraperitoneally (ip) by complete clamping the trachea at electrocardiogram (EGC) registration. The antihypoxic effect was evaluated by duration of heart bioelectric activity (HBA). This model allows to estimate the heart sensitivity to hypoxia. An isoelectric ECG line for 1 minute considered as HBA termination, the pint of HBA termination corresponded to the last QRS complex on the ECG. The calculation of antitoxic activity was performed by the above formula considering a point of last QRS complex registration as a lifetime.

The preliminary screening of test substances was performed on ANHHH model. All derivatives were administered in the same dose of 10 mg/kg intraventricularly (iv) 1 hour prior to pathological state modeling. The effect of substances that were found as the most active by the results of previous tests was studied in HBA model. The effectiveness of a leader compound was evaluated at doses 5; 10 and 15 mg/kg iv. Mexydol was chosen as a reference drug having antihypoxic action in combination with antioxidant and membrane protective activity, which is successfully used in patients with CVA and IM. Mexydol was administered intraperitoneally (ip) at a dose of 100 mg/kg according to similar scheme.

### (10) Example 27

### Assessment of Pro/Antiapoptotosis Properties of 3,2i-spiro-pyrrolo-2-oxindole Derivatives

The assessment of pro/antiapoptotosis properties of substances was carried in intact male rats of Wistar line. The test substance were administered for 2 weeks by means of a probe in aqueous suspension containing Tween-80 at a dose of 50 mg/kg of body weight. The animals were euthanized by cervical vertebrae translocation 24 hours after the experiment. The identification of apoptosis of liver and pancrea cells was performed using electrophoretic method [75]. Ectrophoregrams showed apoptotic DNA fragmentation as a “ladder” of DNA fragments of different lengths. Cell necrosis conditioned the “smeared” nature of DNA migration zone. The luminescence band of intact DNA was in the starting area.

One of the universal apoptosis inducers is oxidative stress associated with reactogenic oxygen metabolites such accumulating in cells under different effects, especially on the background of antioxidant system activity suppression. In view of leading role of oxidative stress in the stroke pathogenesis it would be feasible to study pro/antiapoptotosis properties of 3,2′-spiro-pyrrolo-2-oxindole derivatives. Apoptosis can occur also as a response to endogenous factors—hormones, cytokines, arahidonic acid deprivates, and direct intercellular contacts. Most of the factors that cause cell death are able to induce apoptosis when acting in small doses.

In recent years, information appeared in literature about the ability of antioxidants to prevent free radical DNA oxidation, chromatin proteins and DNA repair enzymes, and inhibit cell death.

It is well known that one of forms of cell response to stressing action, which would be caused by DNA other structural cell element damages, lack of required factors of level growth for hormone, cytokines, etc., is the activation of cells suicidal program—apoptosis. The relevance of apoptosis problem is determined by connectivity of its regulation defects with a wide range of diseases, including diabetes mellitus type 2. The accumulation in the cell of reactive oxygen species precedes apoptosis, indicating the significant importance of oxidation processes in this phenomenon occurrence.

This mechanism is known to be caused by different signals: binding to receptors of specific killer ligands, lack of growth/survival factors, DNA damages and cytoskeleton destruction, hypoxia and other adverse conditions. Then these fragments usually decompose into nucleosomes and their oligomers. Apoptosis—in contrast to necrosis—is never accompanied by inflammatory reaction that also complicates its histological detection. The chromatin condensation is characteristic apoptosis manifestation. Chromatin condenses on the periphery, under nucleus membrane, with clearly delineated dense masses of different shapes and sizes. The nucleus can also break into two or more fragments. The mechanism of chromatin condensation is studied well enough. It is caused by cleavage of nuclear DNA in sites connecting individual nucleosomes resulting in development of large number of fragments in which the number of pairs is divided to 180-200. At electrophoresis said fragments give the characteristic “ladders” pattern. This pattern differs from that in cell necrosis, wherein the length of DNA fragments varies. The fragmentation of DNA in the nucleosome occurs under the influence of calcium-sensitive endonuclease. Endonuclease reside in some cells (for example in thymocytes), wherein it is activated by the appearance of free calcium in cytoplasm and in other cells it is synthesized before apoptosis.

The expression of apoptosis manifestations was assessed by registration of DNA fragments via electrophoresis.

The assessment of pro/antiapoptosis properties was conducted in groups of control rats and rats that once consumed the following substances derivatives of 3,2′-spiro-pyrrolo-2-oxindole: SI-86, SI-81, SI-149, SI-34, SI-180F, SI-183F, SI-73N, SI-87-6V, SI-148N, SI-108, and SI-76-5T.

In groups that received the substances SI-86, and SI-34, apoptosis appeared at level of formation of high-molecular weight 7000-4000 bp fragment, suggesting the intensity of apoptosis processes intrinsic for a healthy organism (Figure).

In the group of animals that received the substances SI-108, and SI-76-5T, apoptosis was verified by the presence of fragment 2500-1500 bp, indicating the intensification of DNA degradation. Meanwhile in animals taken the substances SI-81, SI-149 and SI-148N, apoptosis was identified by 1000-500 bp fragments, which supports the existence of greater apoptosis degree. Under the conditions of consumption be experimental animals of substances SI-180F, SI-183F, SI-173N and SI-87-6V, maximal DNA cleavage was found in this study, namely to fragments 500-200 bp, that indicates the presence of the most expressive pro-apoptosis properties of above compounds among evaluated substances with fluoro and bromo radicals (Figure).

It is possible that free radicals are able to interact both directly with DNA nitrogenous bases, forming their modified derivatives, in particular aminohypoxantine, and indirectly via secondary and end products of lipid peroxidation (malonic dialdehyde and its derivatives) that can bind to DNA and nuclear chromatin proteins, resulting in distortion of the processes of genetic information—replication and transcription reading.

In this context it is interesting to have information about existence in nuclear chromatin of independent system for reoxidation of chromatin-bound lipid at modification of reactions for formation of the same free radicals directly interacting with DNA and chromatin proteins resulting in damage thereof.

In rats treated with substances SI-34 and SI-86 the level of apoptosis was found to be closed to that of control group, probably these substances stimulate the body antioxidant protective system, which controls and inhibits all phases of free radical reactions, starting from their initiation and till formation of hydroperoxides and MDA. The main control mechanism of these reactions is associated with a chain of reversible redox reactions of metal ions, glutathione, ascorbate, tocopherol and other substances of particular importance for preservation of long-lived macromolecules of nucleic acids and proteins, and certain membrane components.

### Example 28

Aqueous solution of SI-86 compound or one of pharmaceutically accepted salts thereof was prepared as follows. SI-86 compound or one of pharmaceutically accepted salts thereof was dissolved in water for injection at agitation without heating. The resulting solution was ampoloued and sterilized by autoclaving at 121° C. for 15 minutes.

### Example 29

Tablets of SI-86 compound or one of pharmaceutically accepted salts thereof were prepared as follows. SI-86 compound or one of pharmaceutically accepted salts thereof mixed with a filler (for example microcrystalline cellulose), disintegrator (for example croscarmellose) and powdering agent (for example calcium stearate). Prepared mixture was stirred for 20 minutes and tableted on tablet press at a rate of 75 000 units per hours.

### Example 30

Syrup of SI-86 compound or one of pharmaceutically accepted salts thereof prepared as follows. SI-86 compound or one of pharmaceutically accepted salts thereof was dissolved in treated water while stirring without heating. To the resulting solution flavor, corrective agent, preservative agent and thickener were added. It was heated to 60° C. and stirred for 30 minutes. The resulted syrup was poured on bottles and sealed.

1-15. (canceled)

16. Spirocyclic compound based on 2-oxindole derivatives containing spiro[indolo-3,1′-pyrrolo[3,4-c]-pyrrol] core and remainders of biogenic sulphur-containing aminoacids of general Formula I 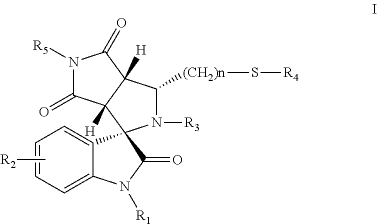
wherein:
R₁ is H, Me-, Et-, Allyl- or -Bn;
R₂ is H, 5-Me, 5-F, 5-Br, -5-OCF₃ or 5-NO₂;
R₃ is H or —N═O;
R₄ is residuals of biogenic sulphur-containing aminoacids, selected from methionine (n=2, R₄=Me), ethionine (n=2, R₄=Et), cysteine (n=1, R₄=H) or cysteine alkyl-derivatives, wherein R₄=Bn or —CH₂CO₂Et, or Alyl-;
R₅ is H, or remainders of Ar, wherein Ar is p-Tolyl, m-Tolyl, 2-(HO)Ph-, 3-(HO)Ph-, 4-Br-Ph-;
4-NO₂-Ph-; 2-NO₂-Ph-; 2-Br-Ph- or 4-(HOOC)Ph-, or pharmaceutically acceptable salt, solvate, hydrate or enantiomer thereof.

17. Spirocyclic compound according to claim 16, wherein said compound is pharmaceutically acceptable salt of general Formula II 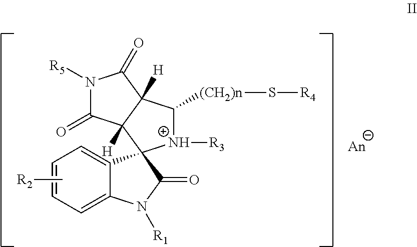
wherein An⁻ is selected from the group consisting of chloride, bromide, iodide, succinate, hemisuccinate, L-aspartate, tartrate or hydrotartrate, nicotinate, L-ascorbate, maleate or hydromaleate, fumarate, hydrofumarate, citrates, L-lactate, L-malate, phosphate, sulphate, benzoate, acetate, pivolate, glutarate, glutamate, asparaginate.

18. Spirocyclic compound according to claim 16, wherein said compound is selected from the group consisting of:
5′-(4-methylphenyl)-3′-[2-(methylthio)ethyl]-3a′,6a′-dihydro-2′H-spiro[indole-3,1′-pyrrolo[3,4-c]pyrrol]-2,4′,6′(1H,3′H,5′H)-trione,
5′-(4-methylphenyl)-3′-[2-(methylthio)ethyl]-3a′,6a′-dihydro-2′H-spiro[indole-3,1′-pyrrolo[3,4-c]pyrrol]-2,4′,6′(1H,3H5′H)-trione,
5′-(4-methylphenyl)-3′-[2-(ethylthio)ethyl]-3a′,6a′-dihydro-2′H-spiro[indole-3,1′-pyrrolo[3,4-c]pyrrol]-2,4′,6′(1H,3′H, 5′H)-trione,
5-6poMo-5′-(4-methylphenyl)-3′-[2-( [symbol] ) [symbol] ]-3a′,6a′-dihydro-2′H-spiro[indole-3,1′-pyrrolo[3,4-c]pyrrol]-2,4′,6′(1H,3′H,5′H)-trione,
5-6poM-5′-(4-methylphenyl)-3′-[2-(methylthio)ethyl]-3a′,6a′-dihydro-2′H-spiro[indole-3,1′-pyrrolo [3,4-c]pyrrol]-2,4′,6′(1H,3′H,5′H)-trione,
1-methyl-5′-(4-methylphenyl)-3′-[2-(methylthio)ethyl]-3a′,6a′-dihydro-2′H-spiro[indole-3,1′-pyrrolo [3,4-c]pyrrol]-2,4′,6′(1H,3′H, 5′H)-trione,
1-(4-chlorobenzyl)-3′-[2-(ethylthio)ethyl]-5′-(4-methylphenyl)-3a′,6a′-dihydro-2′H-spiro[indole-3,1′-pyrrolo[3,4-c]pyrrol]-2,4′,6′(1H,3′H,5′H)-trione,
5-6poM-3′-[2-(ethylthio)ethyl]-3a′,6a′-dihydro-2′H-spiro[indolo-3,1′-pyrrolo[3,4-c]pyrrol]-2,4′,6′(1H,3′H, 5′H)-trione,
1-allyl-3′-[2-(meTiπthio)ethyl]-3a′,6a′-dihydro-2′H-spiro[indole-3,1′-pyrrolo[3,4-c]pyrrol]-2,4′,6′(1H,3′H, 5′H)-trione,
1-allyl-3′-(mercaptomethylen)-3a′,6a′-dihydro-2′H-spiro[indole-3,1′-pyrrolo[3,4-c]pyrrol]-2,4′,6′(1H,3′H, 5′H)-trione,
1-methyl-3′-(mercaptomethylen)-3a′,6a′-dihydro-2′H-spiro[indole-3,1′-pyrrolo[3,4-c]pyrrol]-2,4′,6′(1H,3′H, 5′H)-trione,
3′-(mercaptomethylen)-3a′,6a′-dihydro-2′H-spiro[indole-3,1′-pyrrolo[3,4-c]pyrrol]-2,4′,6′(1H,3′H, 5′H)-trione,
1-allyl-3′-[2-(ethylthio)ethyl]-5′-(4-methylphenyl)-3a′,6a′-dihydro-2′H-spiro[indole-3,1′-pyrrolo[3,4-c]pyrrol]-2,4′,6′(1H,3′H,5′H)-trione,
5-fluoro-3′-[2-(ethylthio)ethyl]-5′-(4-methylphenyl)-3a′,6a′-dihydro-2′H-spiro[indolo-3,1′-pyrrolo[3,4-c]pyrrol]-2,4′,6′(1H,3′H,5′H)-trione,
5-fluoro-3′-[2-(ethylthio)ethyl]-3a′,6a′-dihydro-2′H-spiro[indolo-3,1′-pyrrolo[3,4-c]pyrrol]-2,4′,6′(1H,3′H, 5′H)-trione,
5-bromo-3′-[2-(methylthio)ethyl]-3a′,6a′-dihydro-2′H-spiro[indolo-3,1′-pyrrolo[3,4-c]pyrrol]-2,4′,6′(1H,3′H,5′H)-trione,
3′-[2-(methylthio)ethyl]-3a′,6a′-dihydro-2′H-spiro[indolo-3,1′-pyrrolo[3,4-c]pyrrol]-2,4′,6′(1H,3′H, 5′H)-trione,
1-methyl-3′-[2-(ethylthio)ethyl]-3a′,6a′-dihydro-2′H-spiro[indolo-3,1′-pyrrolo[3,4-c]pyrrol]-2,4′,6′(1H,3′H, 5′H)-trione.

19. Spirocyclic compound of claim 16, wherein said compound exhibits glucocorticoidomodeling activity.

20. Spirocyclic compound of claim 16, wherein said compound exhibits antioxidant, antihypoxant, cerebroprotective or cytoprotective action.

21. The use of compounds of claim 16 for treatment of diseases associated with cortisol production.

22. The use according to claim 21, wherein said disease is selected from the group consisting of stroke, traumatic brain injury, chronic cerebrovascular pathology, Alzheimer's disease, encephalopathy, diabetes mellitus, retinodegenerative eye diseases, metabolic syndrome, adiposity, Cushing syndrome, metabolic Riven syndrome, insulin resistance; hyperglycemia; hypertension; hyperlipidemia; cognitive impairments; depression; dementia, glaucoma; cardiovascular diseases; osteoporosis; inflammation; excess of androgenic hormones or polycystic ovary syndrome (PCOS).

23. A pharmaceutical composition containing the compound of claim 16 as an active agent and pharmaceutically acceptable carrier.

24. The pharmaceutical composition according to claim 23, wherein said composition is made in a form selected from the group, comprising tablets, pills, powders, lozenges, sachets, suspensions, emulsions, solutions for oral administration, syrups, aerosols, dispersions, ointments, drops, soft or hard gelatin capsules, suppositories, solutions for injection, and infusions.

25. The pharmaceutical composition according to claim 23, wherein said composition is intended for treatment of a disease selected from the group consisting in stroke, traumatic brain injury, chronic cerebrovascular pathology, Alzheimer's disease, encephalopathy, diabetes mellitus, retinodegenerative eye diseases, metabolic syndrome, adiposity, Cushing syndrome, metabolic Riven syndrome, insulin resistance; hyperglycemia; hypertension; hyperlipidemia; cognitive impairments; depression; dementia, glaucoma; cardiovascular diseases; osteoporosis; inflammation; excess of androgenic hormones or polycystic ovary syndrome (PCOS).

26. The pharmaceutical composition according to claim 23, wherein single dose of compounds of any of claims 1-3 is from 0.25 to 50 mg per kg body weight.

27. A process for preparation of compounds according to claim 16, comprising the two-stage synthesis based on three-component enantioselective condensation reaction.

28. The process for preparation according to claim 27, comprising one-stage condensation of corresponding pyrrol-2,5-dions with 1H-indole-2,3-dions and biogenic sulphur-containing aminoacids in environment of methyl or isopropyl, or ethyl alcohols, or acetonitrile in mixture with water in the ratio range of from 2:1 to 10:1.

29. The process for preparation according to claim 27, wherein the most appropriate ratio is the ratio of 3:1.

30. The process for preparation of compounds according to claim 27, comprising the dissolution of a corresponding base of compound in ethanol, wherein said base of compound comprises a spirocyclic compound based on 2-oxindole derivatives containing spiro[indolo-3,1′-pyrrolo[3,4-c]-pyrrol] core and remainders of biogenic sulphur-containing aminoacids of general Formula I 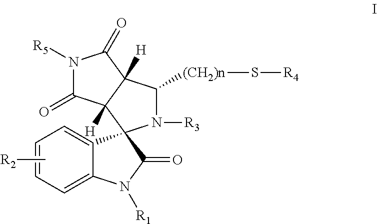
wherein:
R₁ is H, Me-, Et-, Allyl- or -Bn;
R₂ is H, 5-Me, 5-F, 5-Br, -5-OCF₃ or 5-NO₂;
R₃ is H or —N═O;
R₄ is residuals of biogenic sulphur-containing aminoacids, selected from methionine (n=2, R₄=Me), ethionine (n=2, R₄=Et), cysteine (n=1, R₄=H) or cysteine alkyl-derivatives, wherein R₄=Bn or —CH₂CO₂Et, or Alyl-;
R₅ is H, or remainders of Ar, wherein Ar is p-Tolyl, m-Tolyl, 2-(HO)Ph-, 3-(HO)Ph-, 4-Br-Ph-;
4-NO₂-Ph-; 2-NO₂-Ph-; 2-Br-Ph- or 4-(HOOC)Ph-, or pharmaceutically acceptable salt, solvate, hydrate or enantiomer thereof.

31. The process for preparation of compounds according to claim 27, comprising the mixing ethanol with water, or butanol, and adding aqueous or alcoholic solution of a corresponding organic or inorganic, followed by evaporation in vacuum, wherein the corresponding organic or inorganic comprises a pharmaceutically acceptable salt of general Formula II 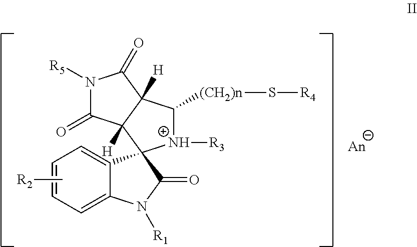
wherein An⁻ is selected from the group consisting of chloride, bromide, iodide, succinate, hemisuccinate, L-aspartate, tartrate or hydrotartrate, nicotinate, L-ascorbate, maleate or hydromaleate, fumarate, hydrofumarate, citrates, L-lactate, L-malate, phosphate, sulphate, benzoate, acetate, pivolate, glutarate, glutamate, asparaginate.
